SEQUENCE LISTING (17 sequences)

SEQ ID NO: 1 - DNA, 5678 bp, Homo sapiens
aggcgaaggcttggacccccagctgaggagtcctgctcaagacacggtcactggatctgagaaacttcccaggggaccgcattccagagtcagtgactctgtgaagcacccacatctacctcttgccacgttcccacgggcttgggggaaagatggtggggaccaaggcctgggtgttctccttcctggtcctggaagtcacatctgtgttggggagacagacgatgctcacccagtcagtaagaagagtccagcctgggaagaagaaccccagcatctttgccaagcctgccgacaccctggagagccctggtgagtggacaacatggttcaacatcgactacccaggcgggaagggcgactatgagcggctggacgccattcgcttctactatggggaccgtgtatgtgcccgtcccctgcggctagaggctcggaccactgactggacacctgcgggcagcactggccaggtggtccatggtagtccccgtgagggtttctggtgcctcaacagggagcagcggcctggccagaactgctctaattacaccgtacgcttcctctgcccaccaggatccctgcgccgagacacagagcgcatctggagcccatggtctccctggagcaagtgctcagctgcctgtggtcagactggggtccagactcgcacacgcatttgcttggcagagatggtgtcgctgtgcagtgaggccagcgaagagggtcagcactgcatgggccaggactgtacagcctgtgacctgacctgcccaatgggccaggtgaatgctgactgtgatgcctgcatgtgccaggacttcatgcttcatggggctgtctcccttcccggaggtgccccagcctcaggggctgctatctacctcctgaccaagacgccgaagctgctgacccagacagacagtgatgggagattccgaatccctggcttgtgccctgatggcaaaagcatcctgaagatcacaaaggtcaagtttgcccccattgtactcacaatgcccaagactagcctgaaggcagccaccatcaaggcagagtttgtgagggcagagactccatacatggtgatgaaccctgagacaaaagcacggagagctgggcagagcgtgtctctgtgctgtaaggccacagggaagcccaggccagacaagtatttttggtatcataatgacacattgctggatccttccctctacaagcatgagagcaagctggtgctgaggaaactgcagcagcaccaggctggggagtacttttgcaaggcccagagtgatgctggggctgtgaagtccaaggttgcccagctgattgtcatagcatctgatgagactccttgcaacccagttcctgagagctatcttatccggctgccccatgattgctttcagaatgccaccaactccttctactatgacgtgggacgctgccctgttaagacttgtgcagggcagcaggataatgggatcaggtgccgtgatgctgtgcagaactgctgtggcatctccaagacagaggaaagggagatccagtgcagtggctacacgctacccaccaaggtggccaaggagtgcagctgccagcggtgtacggaaactcggagcatcgtgcggggccgtgtcagtgctgctgacaatggggagcccatgcgctttggccatgtgtacatggggaacagccgtgtaagcatgactggctacaagggcactttcaccctccatgtcccccaggacactgagaggctggtgctcacatttgtggacaggctgcagaagtttgtcaacaccaccaaagtgctacctttcaacaagaaggggagtgccgtgttccatgaaatcaagatgcttcgtcggaaaaagcccatcactttggaagccatggagaccaacatcatccccctgggggaagtggttggtgaagaccccatggctgaactggagattccatccaggagtttctacaggcagaatggggagccctacataggaaaagtgaaggccagtgtgaccttcctggatccccggaatatttccacagccacagctgcccagactgacctgaacttcatcaatgacgaaggagacactttcccccttcggacgtatggcatgttctctgtggacttcagagatgaggtcacctcagagccacttaatgctggcaaagtgaaggtccaccttgactcgacccaggtcaagatgccagagcacatatccacagtgaaactctggtcactcaatccagacacagggctgtgggaggaggaaggtgatttcaaatttgaaaatcaaaggaggaacaaaagagaagacagaaccttcctggtgggcaacctggagattcgtgagaggaggctctttaacctggatgttcctgaaagcaggcggtgctttgttaaggtgagggcctaccggagtgagaggttcttgcctagtgagcagatccagggggttgtgatctccgtgattaacctggagcctagaactggcttcttgtccaaccctagggcctggggccgctttgacagtgtcatcacaggccccaacggggcctgtgtgcctgccttctgtgatgaccagtcccctgatgcctactctgcctatgtcttggcaagcctggctggggaggaactgcaagcagtggagtcttctcctaaattcaacccaaatgcaattggcgtccctcagccctatctcaacaagctcaactaccgtcggacggaccatgaggatccacgggttaaaaagacagctttccagattagcatggccaagccaaggcccaactcagctgaggagagcaatgggcccatctatgcctttgagaacctccgggcatgtgaagaggcaccacccagtgcagcccacttccggttctaccagattgagggggatcgatatgactacaacacagtccccttcaacgaagatgaccctatgagctggactgaagactatctggcatggtggccaaagccgatggaattcagggcctgctatatcaaggtgaagattgtggggccactggaagtgaatgtgcgatcccgcaacatggggggcactcatcggcagacagtggggaagctgtatggaatccgagatgtgaggagcactcgggacagggaccagcccaatgtctcagctgcctgtctggagttcaagtgcagtgggatgctctatgatcaggaccgtgtggaccgcaccctggtgaaggtcatcccccagggcagctgccgtcgagccagtgtgaaccccatgctgcatgagtacctggtcaaccacttgccacttgcagtcaacaacgacaccagtgagtacaccatgctggcacccttggacccactgggccacaactatggcatctacactgtcactgaccaggaccctcgcacggccaaggagatcgcgctcggccggtgctttgatggcacatccgatggctcctccagaatcatgaagagcaatgtgggagtagccctcaccttcaactgtgtagagaggcaagtaggccgccagagtgccttccagtacctccaaagcaccccagcccagtcccctgctgcaggcactgtccaaggaagagtgccctcgaggaggcagcagcgagcgagcaggggtggccagcgccagggtggagtggtggcctctctgagatttcctagagttgctcaacagcccctgatcaactaagttttgtggtacttcaccctcttctgccctcatttcatgtgacagccattgtgagactgatgcacaaactgtcacttggttaatttaagcacttctgttttcgtgaatttgcttgtttgtttcttcatgcctttacttactttgtcccatgctactgattggcacgtggcccccacaatggcacaataaagcccctttgtgaaactgttctttaaatgaaacacaagaaattggccactggtaaaactctgcagcttcaactgtacttcatttaatgccattaatgcaaatatacttcctcttctttttgcatggttttgcccacctctgcaatagtgataatctgatgctgaagatcaaataaccaatataaagcatatttcttggccttgctccacaggacataggcaagccttgatcatagttcatacatataaatggtggtgaaataaagaaataaaacacaatacttttacttgaaatgtaaataacttatttatttctttgctaaatttggaattctagtgcacattcaaagttaagctattaaatatagggtgatcatagttcctctaccaagtctggaaagaacatctcctggtatccacaattacaccaggttgctaactgtatttgtacatttccctttgcattcgcttctgttcttgctagaaacccagtgtagcccagggcagatgtcaataaatgcatactctgtatttcgattctgaggccactgtcctgtcttaactattaaagtgagccagcttctcagaggagtacaggggactctggaaagcagtcacaggtggtgtaagtcctcgccccctaagttacagggcagggcaacataggcacacactccagcagtcaaatttgttctcatggatcacattttccgtttgcagaaaagagcagattcctggagttgtgagcatgttctgggctcctgagcaagactaaaaacacgtcccgatttttttctttccttctttccaatactttggagcttatacaatggcttggcccagggtctgacaagctggccaagtggtcctttggctctcccaacagcctgttgccttctccagctgggctggccggcctcactcagaaatgggcttttacttttttctttatgctcttctaaagatgttcaacttttccagacaccagatctctgttgtgtgttacagaccctgtccctggagcaaaacaattggtccaaacgggcagagttttggttttggaaaaggtatttggtttaaaatagtaaatcagaggcccataggtggtgccagagcgatctcagcggcattacagtcagggtagtggggtgtggggttggataggatgcactcactgcccctatcccaacccctccagaatctgtcaggcaagagtccaaagggtttgggagaagagaggacactgtttacaatcaggacaaaaagctttccctgatgccctggccagcctagtgcagctcctttacccatctctggcctgggctggatttgtactcttggatcaccatctgcccggctcctgggaatttggtttgtaagcaattagaaatttctctagctgagcacatcacaataaccatagaaaaagcaagcatctgcaggagtgaggactagtggtcttgtgattgcaacttgacccacttccagactccacatctgtcccactgctcccaggaagcagctctcactgactgcttggggaggcttgactgcccctgttgtgagtgcacagctatctgcagcaccatcgccccagccacttcaggagccagagtgcctgctgcttcaacctgaccaccagttcacttcaaactcacggggggagggggagggtcgggggggggtgggttgctggggaaggtgggctgactggttccccactctagggcactttccttgctgttctcaattgagggcctgaataaatggactccagcacaca

SEQ ID NO: 2 - protein, 1184 aa, Homo sapiens
MVGTKAWVFSFLVLEVTSVLGRQTMLTQSVRRVQPGKKNPSIFAKPADTLESPGEWTTWFNIDYPGGKGDYERLDAIRFYYGDRVCARPLRLEARTTDWTPAGSTGQVVHGSPREGFWCLNREQRPGQNCSNYTVRFLCPPGSLRRDTERIWSPWSPWSKCSAACGQTGVQTRTRICLAEMVSLCSEASEEGQHCMGQDCTACDLTCPMGQVNADCDACMCQDFMLHGAVSLPGGAPASGAAIYLLTKTPKLLTQTDSDGRFRIPGLCPDGKSILKITKVKFAPIVLTMPKTSLKAATIKAEFVRAETPYMVMNPETKARRAGQSVSLCCKATGKPRPDKYFWYHNDTLLDPSLYKHESKLVLRKLQQHQAGEYFCKAQSDAGAVKSKVAQLIVIASDETPCNPVPESYLIRLPHDCFQNATNSFYYDVGRCPVKTCAGQQDNGIRCRDAVQNCCGISKTEEREIQCSGYTLPTKVAKECSCQRCTETRSIVRGRVSAADNGEPMRFGHVYMGNSRVSMTGYKGTFTLHVPQDTERLVLTFVDRLQKFVNTTKVLPFNKKGSAVFHEIKMLRRKKPITLEAMETNIIPLGEVVGEDPMAELEIPSRSFYRQNGEPYIGKVKASVTFLDPRNISTATAAQTDLNFINDEGDTFPLRTYGMFSVDFRDEVTSEPLNAGKVKVHLDSTQVKMPEHISTVKLWSLNPDTGLWEEEGDFKFENQRRNKREDRTFLVGNLEIRERRLFNLDVPESRRCFVKVRAYRSERFLPSEQIQGVVISVINLEPRTGFLSNPRAWGRFDSVITGPNGACVPAFCDDQSPDAYSAYVLASLAGEELQAVESSPKFNPNAIGVPQPYLNKLNYRRTDHEDPRVKKTAFQISMAKPRPNSAEESNGPIYAFENLRACEEAPPSAAHFRFYQIEGDRYDYNTVPFNEDDPMSWTEDYLAWWPKPMEFRACYIKVKIVGPLEVNVRSRNMGGTHRQTVGKLYGIRDVRSTRDRDQPNVSAACLEFKCSGMLYDQDRVDRTLVKVIPQGSCRRASVNPMLHEYLVNHLPLAVNNDTSEYTMLAPLDPLGHNYGIYTVTDQDPRTAKEIALGRCFDGTSDGSSRIMKSNVGVALTFNCVERQVGRQSAFQYLQSTPAQSPAAGTVQGRVPSRRQQRASRGGQRQGGVVASLRFPRVAQQPLIN

SEQ ID NO: 3 - DNA, 2769 bp, Homo sapiens
cgacgccaaggggagggggctgacctgtgcttggtccaccccaggtaggggctgagagaggcttgaggtggaagtgggggtcgggcactctgacctggtcgaggaggggctagggtttgaaccggggacagagtctaggtgagctggggcttgggagctattagcgtagaggatccgggttcggttgctctggcgagggctccagcatcacaggcggcggctgcgggcgcagagcggagatgcagcggcttggggccaccctgctgtgcctgctgctggcggcggcggtccccacggcccccgcgcccgctccgacggcgacctcggctccagtcaagcccggcccggctctcagctacccgcaggaggaggccaccctcaatgagatgttccgcgaggttgaggaactgatggaggacacgcagcacaaattgcgcagcgcggtggaagagatggaggcagaagaagctgctgctaaagcatcatcagaagtgaacctggcaaacttacctcccagctatcacaatgagaccaacacagacacgaaggttggaaataataccatccatgtgcaccgagaaattcacaagataaccaacaaccagactggacaaatggtcttttcagagacagttatcacatctgtgggagacgaagaaggcagaaggagccacgagtgcatcatcgacgaggactgtgggcccagcatgtactgccagtttgccagcttccagtacacctgccagccatgccggggccagaggatgctctgcacccgggacagtgagtgctgtggagaccagctgtgtgtctggggtcactgcaccaaaatggccaccaggggcagcaatgggaccatctgtgacaaccagagggactgccagccggggctgtgctgtgccttccagagaggcctgctgttccctgtgtgcacacccctgcccgtggagggcgagctttgccatgaccccgccagccggcttctggacctcatcacctgggagctagagcctgatggagccttggaccgatgcccttgtgccagtggcctcctctgccagccccacagccacagcctggtgtatgtgtgcaagccgaccttcgtggggagccgtgaccaagatggggagatcctgctgcccagagaggtccccgatgagtatgaagttggcagcttcatggaggaggtgcgccaggagctggaggacctggagaggagcctgactgaagagatggcgctgagggagcctgcggctgccgccgctgcactgctgggaggggaagagatttagatctggaccaggctgtgggtagatgtgcaatagaaatagctaatttatttccccaggtgtgtgctttaggcgtgggctgaccaggcttcttcctacatcttcttcccagtaagtttcccctctggcttgacagcatgaggtgttgtgcatttgttcagctcccccaggctgttctccaggcttcacagtctggtgcttgggagagtcaggcagggttaaactgcaggagcagtttgccacccctgtccagattattggctgctttgcctctaccagttggcagacagccgtttgttctacatggctttgataattgtttgaggggaggagatggaaacaatgtggagtctccctctgattggttttggggaaatgtggagaagagtgccctgctttgcaaacatcaacctggcaaaaatgcaacaaatgaattttccacgcagttctttccatgggcataggtaagctgtgccttcagctgttgcagatgaaatgttctgttcaccctgcattacatgtgtttattcatccagcagtgttgctcagctcctacctctgtgccagggcagcattttcatatccaagatcaattccctctctcagcacagcctggggagggggtcattgttctcctcgtccatcagggatctcagaggctcagagactgcaagctgcttgcccaagtcacacagctagtgaagaccagagcagtttcatctggttgtgactctaagctcagtgctctctccactaccccacaccagccttggtgccaccaaaagtgctccccaaaaggaaggagaatgggatttttcttttgaggcatgcacatctggaattaaggtcaaactaattctcacatccctctaaaagtaaactactgttaggaacagcagtgttctcacagtgtggggcagccgtccttctaatgaagacaatgatattgacactgtccctctttggcagttgcattagtaactttgaaaggtatatgactgagcgtagcatacaggttaacctgcagaaacagtacttaggtaattgtagggcgaggattataaatgaaatttgcaaaatcacttagcagcaactgaagacaattatcaaccacgtggagaaaatcaaaccgagcagggctgtgtgaaacatggttgtaatatgcgactgcgaacactgaactctacgccactccacaaatgatgttttcaggtgtcatggactgttgccaccatgtattcatccagagttcttaaagtttaaagttgcacatgattgtataagcatgctttctttgagttttaaattatgtataaacataagttgcatttagaaatcaagcataaatcacttcaactgctcttctgtagttcttggatttcttttcccttttgactttgaataaatgtaaaatcctttcagccagaaaaagtaaaatagaaacaacctgtattaaaaatcttccatagaaaaaaaaaaa

SEQ ID NO: 4 - protein, 350 aa, Homo sapiens
MQRLGATLLCLLLAAAVPTAPAPAPTATSAPVKPGPALSYPQEEATLNEMFREVEELMEDTQHKLRSAVEEMEAEEAAAKASSEVNLANLPPSYHNETNTDTKVGNNTIHVHREIHKITNNQTGQMVFSETVITSVGDEEGRRSHECIIDEDCGPSMYCQFASFQYTCQPCRGQRMLCTRDSECCGDQLCVWGHCTKMATRGSNGTICDNQRDCQPGLCCAFQRGLLFPVCTPLPVEGELCHDPASRLLDLITWELEPDGALDRCPCASGLLCQPHSHSLVYVCKPTFVGSRDQDGEILLPREVPDEYEVGSFMEEVRQELEDLERSLTEEMALREPAAAAAALLGGEEI

SEQ ID NO: 5 - DNA, 1996 bp, Homo sapiens
gccccagcaacggctcattctgctcccccgggtcggagccccccggagctgcgcgcgggcttgcagcgcctcgcccgcgctgtcctcccggtgtcccgcttctccgcgccccagccgccggctgccagcttttcggggccccgagtcgcacccagcgaagagagcgggcccgggacaagctcgaactccggccgcctcgcccttccccggctccgctccctctgccccctcggggtcgcgcgcccacgatgctgcagggccctggctcgctgctgctgctcttcctcgcctcgcactgctgcctgggctcggcgcgcgggctcttcctctttggccagcccgacttctcctacaagcgcagcaattgcaagcccatccctgccaacctgcagctgtgccacggcatcgaataccagaacatgcggctgcccaacctgctgggccacgagaccatgaaggaggtgctggagcaggccggcgcttggatcccgctggtcatgaagcagtgccacccggacaccaagaagttcctgtgctcgctcttcgcccccgtctgcctcgatgacctagacgagaccatccagccatgccactcgctctgcgtgcaggtgaaggaccgctgcgccccggtcatgtccgccttcggcttcccctggcccgacatgcttgagtgcgaccgtttcccccaggacaacgacctttgcatccccctcgctagcagcgaccacctcctgccagccaccgaggaagctccaaaggtatgtgaagcctgcaaaaataaaaatgatgatgacaacgacataatggaaacgctttgtaaaaatgattttgcactgaaaataaaagtgaaggagataacctacatcaaccgagataccaaaatcatcctggagaccaagagcaagaccatttacaagctgaacggtgtgtccgaaagggacctgaagaaatcggtgctgtggctcaaagacagcttgcagtgcacctgtgaggagatgaacgacatcaacgcgccctatctggtcatgggacagaaacagggtggggagctggtgatcacctcggtgaagcggtggcagaaggggcagagagagttcaagcgcatctcccgcagcatccgcaagctgcagtgctagtcccggcatcctgatggctccgacaggcctgctccagagcacggctgaccatttctgctccgggatctcagctcccgttccccaagcacactcctagctgctccagtctcagcctgggcagcttccccctgccttttgcacgtttgcatccccagcatttcctgagttataaggccacaggagtggatagctgttttcacctaaaggaaaagcccacccgaatcttgtagaaatattcaaactaataaaatcatgaatatttttatgaagtttaaaaatagctcactttaaagctagttttgaataggtgcaactgtgacttgggtctggttggttgttgtttgttgttttgagtcagctgattttcacttcccactgaggttgtcataacatgcaaattgcttcaattttctctgtggcccaaacttgtgggtcacaaaccctgttgagataaagctggctgttatctcaacatcttcatcagctccagactgagactcagtgtctaagtcttacaacaattcatcattttataccttcaatgggaacttaaactgttacatgtatcacattccagctacaatacttccatttattagaagcacattaaccatttctatagcatgatttcttcaagtaaaaggcaaaagatataaattttataattgacttgagtactttaagccttgtttaaaacatttcttacttaacttttgcaaattaaacccattgtagcttacctgtaatatacatagtagtttacctttaaaagttgtaaaaatattgctttaaccaacactgtaaatatttcagataaacattatattcttgtatataaactttacatcctgttttaccta

SEQ ID NO: 6 - protein, 295 aa, Homo sapiens
MLQGPGSLLLLFLASHCCLGSARGLFLFGQPDFSYKRSNCKPIPANLQLCHGIEYQNMRLPNLLGHETMKEVLEQAGAWIPLVMKQCHPDTKKFLCSLFAPVCLDDLDETIQPCHSLCVQVKDRCAPVMSAFGFPWPDMLECDRFPQDNDLCIPLASSDHLLPATEEAPKVCEACKNKNDDDNDIMETLCKNDFALKIKVKEITYINRDTKIILETKSKTIYKLNGVSERDLKKSVLWLKDSLQCTCEEMNDINAPYLVMGQKQGGELVITSVKRWQKGQREFKRISRSIRKLQC

SEQ ID NO: 7 - DNA, 1823 bp, Homo sapiens
ctgggccgcggctacccagagtcagggccacggagcttaggagaccttgggtcagctctgcaaaggggttgtttactgaatgcttctggtatctgcagccccgctggggttcggacagggggtctggaatagggcttcacaactacagcagggctaggaccctcttgggctggaaggagctggagaggcctctgcaagctgtcacaggctcttggtgctggcactggctcaggctttcacacacacacacgcgcacacacacacacacacacacacggacaggcacccccttggtggccttcacagtttcaccttcaggtaaatgggctcatcctttgagccatgaggatgggaagcgaagcaaggaatgaaaaagctagtgtgtttgtgtgtgtgtgtgtgtgtgtgtgtgtgtgtgagcgcgcgcgcgcgcgcgcgtgtgtactcgtgcgtgtgcctgtgtgtgcctgggagtgacctcacagctgccggaacataaagactcacaggtccgcctcccaggctcaaagctggctctgcaggggacatgagaggcacaccgaagacccacctcctggccttctccctcctctgcctcctctcaaaggtgcgtacccagctgtgcccgacaccatgtacctgcccctggccacctccccgatgcccgctgggagtacccctggtgctggatggctgtggctgctgccgggtatgtgcacggcggctgggggagccctgcgaccaactccacgtctgcgacgccagccagggcctggtctgccagcccggggcaggacccggtggccggggggccctgtgcctcttggcagaggacgacagcagctgtgaggtgaacggccgcctgtatcgggaaggggagaccttccagccccactgcagcatccgctgccgctgcgaggacggcggcttcacctgcgtgccgctgtgcagcgaggatgtgcggctgcccagctgggactgcccccaccccaggagggtcgaggtcctgggcaagtgctgccctgagtgggtgtgcggccaaggagggggactggggacccagccccttccagcccaaggaccccagttttctggccttgtctcttccctgccccctggtgtcccctgcccagaatggagcacggcctggggaccctgctcgaccacctgtgggctgggcatggccacccgggtgtccaaccagaaccgcttctgccgactggagacccagcgccgcctgtgcctgtccaggccctgcccaccctccaggggtcgcagtccacaaaacagtgccttctagagccgggctgggaatggggacacggtgtccaccatccccagctggtggccctgtgcctgggccctgggctgatggaagatggtccgtgcccaggcccttggctgcaggcaacactttagcttgggtccaccatgcagaacaccaatattaacacgctgcctggtctgtctggatcccgaggtatggcagaggtgcaagacctagtcccctttcctctaactcactgcctaggaggctggccaaggtgtccagggtcctctagcccactccctgcctacacacacagcctatatcaaacatgcacacgggcgagctttctctccgacttcccctgggcaagagatgggacaagcagtcccttaatattgaggctgcagcaggtgctgggctggactggccatttttctgggggtaggatgaagagaaggcacacagagattctggatctcctgctgccttttctggagtttgtaaaattgttcctgaatacaagcctatgcgtgataaaaaaaaaaaaaaaaaa

SEQ ID NO: 8 - protein, 250 aa, Homo sapiens
MRGTPKTHLLAFSLLCLLSKVRTQLCPTPCTCPWPPPRCPLGVPLVLDGCGCCRVCARRLGEPCDQLHVCDASQGLVCQPGAGPGGRGALCLLAEDDSSCEVNGRLYREGETFQPHCSIRCRCEDGGFTCVPLCSEDVRLPSWDCPHPRRVEVLGKCCPEWVCGQGGGLGTQPLPAQGPQFSGLVSSLPPGVPCPEWSTAWGPCSTTCGLGMATRVSNQNRFCRLETQRRLCLSRPCPPSRGRSPQNSAF

SEQ ID NO: 9 - DNA, 8460 bp, Homo sapiens
ccaaaaataaaaccgtccgggtccccttcagacggctgcaggcacagggaggaggcgcgaaggtgcagcagccgtgcgagcccagctggagtaggagcgcggactcgaggctcggggcgcgcagccctcgttccgccgagagccgggcccccagtcggccgcttcagggccccctagactcagagaagctggccgccgggcggggccgggagaacagcccgcgggcgtccagcgtgccgaccacaaagctcttcgcggtgcccgcgcgcaccactctccagccgccccgcgccatgaggccgcggaccaaagcccgcagcccggggcgcgccctgcggaacccctggagaggcttcctgccgctcaccctggctctcttcgtgggcgcgggtcatgcccaaagggaccccgtagggagatacgagccggctggtggagacgcgaatcgactgcggcgccctgggggcagctacccggcagcggctgcagccaaggtgtacagtctgttccgggagcaggacgcgcctgtcgcgggcttgcagcccgtggagcgggcccagccgggctgggggagccccaggaggcccaccgaggcggaggccaggaggccgtcccgcgcgcagcagtcgcggcgtgtccagccacctgcgcagacccggagaagcactcccctgggccagcagcaaccagcaccccggacccgggccgcgccggctctcccacgcctggggaccccacagcggtctggggctgcgcccccaaccccgccgcgagggcggctcacggggaggaacgtctgcgggggacagtgctgcccaggatggacaacagcaaacagcaccaaccactgtatcaaacccgtttgcgagccgccgtgccagaaccggggctcctgcagccgcccgcagctctgtgtctgccgctctggtttccgtggagcccgctgcgaggaggtcattcccgatgaggaatttgacccccagaactccaggctggcacctcgacgctgggccgagcgttcacccaacctgcgcaggagcagtgcggctggagagggcaccttggccagagcacagccgccagcaccacagtcgccgcccgcaccacagtcgccaccagctgggaccctgagtggcctcagccagacccacccttcccagcagcacgtggggttgtcccgcactgtccgacttcacccgactgccacggccagtagccagctctcttccaacgccctgcccccgggaccaggccttgagcagagagatggcacccaacaggcggtacctctggagcacccctcatccccctgggggctgaacctcacggagaaaatcaagaagatcaagatcgtcttcactcccaccatctgcaagcagacctgtgcccgtggacactgtgccaacagctgtgagaggggcgacaccaccaccctgtacagccagggcggccatgggcacgatcccaagtctggcttccgcatctatttctgccagatcccctgcctgaacggaggccgctgcatcggcagggacgaatgctggtgccccgccaactccaccgggaagttctgccacctgcctatcccgcagccggacagggagcctccagggagggggtcccgccccagggccttgctggaagccccactgaagcagtccactttcacactgccgctctccaaccagctggcctccgtgaacccctccctggtgaaggtgcacattcaccacccacccgaggcctcagtgcagatccaccaggtggcccaggtgcggggcggggtggaggaggccctagtggagaacagcgtggagaccagacccccgccctggctgcctgccagccctggccacagcctctgggacagcaacaacatccctgctcggtctggagagccccctcggccactgcccccagcagcacccaggcctcgaggactgctgggccggtgttacctgaacactgtgaacggacagtgtgccaaccctctgctggagctgactacccaggaggactgctgtggcagtgtgggagccttctggggggtgactttgtgtgccccatgcccacccagaccagcctccccggtgattgagaatggccagctggagtgtcctcaggggtacaagagactgaacctcactcactgccaagatatcaacgagtgcttgaccctgggcctgtgcaaggacgcggagtgtgtgaataccaggggcagctacctgtgcacatgcagacctggcctcatgctggatccatcgcggagccgctgtgtgtcggacaaggcaatctccatgctgcagggactgtgctaccggtcgctggggcccggcacctgcaccctgcctttggcccagcggatcaccaagcagatatgctgctgcagccgcgtgggcaaagcatggggcagcgagtgtgagaaatgccctctgcctggcacagaggccttcagagagatctgccctgccggccacggctacacctacgcgagctccgacatccgcctgtccatgaggaaagccgaggaggaggaactggcaaggcccccaagggagcaagggcagaggagcagcggggcactgcccgggccagcagagaggcagcccctccgggtcgtcacggacacctggcttgaggccgggaccatccctgacaagggtgactctcaggctggccaggtcacgaccagtgtcactcatgcacctgcctgggtcacagggaatgccacaaccccaccaatgcctgaacaggggattgcagagatacaggaagaacaagtgaccccctccactgatgtgctggtgaccctgagcaccccaggcattgacagatgcgctgctggagccaccaacgtctgtggccctggaacctgcgtgaacctccccgatggatacagatgtgtctgcagccctggctaccagctgcaccccagccaggcctactgcacagatgacaacgagtgtctgagggacccctgcaagggaaaagggcgctgcatcaaccgcgtggggtcctactcctgcttctgctaccctggctacactctggccacctcaggggcgacacaggagtgtcaagatatcaatgagtgtgagcagccaggggtgtgcagcggggggcagtgcaccaacaccgagggctcgtaccactgcgagtgtgatcagggctacatcatggtcaggaaaggacactgccaagatatcaacgaatgccgtcaccccggtacctgccctgatgggagatgcgtcaattcccctggctcctacacttgtctggcctgtgaggagggctaccggggccagagtgggagctgtgtagatgtgaatgagtgtctgactcccggggtctgtgcccatggaaagtgcaccaacctagaaggctccttcagatgctcttgtgagcagggctatgaggtcacctcagatgagaagggctgccaagatgtggatgagtgtgccagccgggcctcatgccccacaggcctctgcctcaacacggagggctccttcgcctgctctgcctgtgagaacgggtactgggtgaatgaagacggcactgcctgtgaagacctagatgagtgtgccttcccgggagtctgcccctccggagtctgcaccaacacggctggctccttctcctgcaaggactgcgatgggggctaccggcccagccccctgggtgactcctgtgaagatgtggatgaatgtgaagacccccagagcagctgcctgggaggcgagtgcaagaacactgtgggctcctaccagtgcctctgtccccagggcttccagctggccaatggcaccgtgtgtgaggatgtgaatgagtgcatgggggaggagcactgcgcaccccacggcgagtgcctcaacagccacgggtctttcttctgtctgtgcgcgcctggcttcgtcagcgcagaggggggcaccagctgccaggatgtggacgagtgtgccaccacagacccgtgtgtgggagggcactgtgtcaacaccgagggctccttcaactgtctatgtgagactggcttccagccctccccagagagtggagagtgtgtggatattgacgagtgtgaggactatggagacccggtgtgtggcacctggaagtgtgaaaacagccctggctcctaccgctgtgttctgggctgccagcctggcttccacatggccccgaacggagactgcattgacatagacgagtgcgccaacgacaccatgtgtggcagccacggcttctgtgacaacactgatggctccttccgctgcctctgtgaccagggcttcgagatctctccctcaggctgggactgtgtggatgtgaacgagtgtgagcttatgctggcggtatgtggggccgcgctctgtgagaacgtggagggctccttcctgtgcctctgtgccagtgacctggaggagtacgatgcccaggaggggcactgccgcccacggggggctggaggtcagagtatgtctgaggccccaacgggggaccatgccccggcccccacccgcatggactgctactccgggcagaagggccatgcgccctgctccagtgtcctgggccggaacaccacacaggctgaatgctgctgcacccagggcgctagctggggagatgcctgtgacctctgcccgtctgaggactcagctgaattcagcgagatctgccctagtggaaaaggctacattcctgtggaaggagcctggacgtttggacagaccatgtacacagatgcggatgagtgtgtgatattcgggcctggtctctgcccgaacggccggtgcctcaacaccgtgcctggttatgtctgcctgtgcaatcccggcttccactacgatgcttcccacaagaagtgtgaggatcacgatgagtgccaggacctggcctgtgagaatggcgagtgcgtcaacacggagggctccttccactgcttctgcagccccccgctcaccctggacctcagccagcagcgctgcatgaacagcaccagcagcacggaggacctccctgaccacgacatccacatggacatctgctggaaaaaagtcaccaatgatgtgtgcagcgaacccctgcgtgggcaccgcaccacctacacggaatgctgctgccaggacggcgaggcctggagccagcagtgtgctctgtgtcccccgaggagctctgaggtctatgctcagctgtgcaacgtggctcgcattgaggcagagcgggaggccggggtccacttccggccaggctatgagtatggccccgggcccgatgacctgcactacagcatctatggcccagatggggcccccttctacaactacctgggccccgaggacaccgtccctgagcctgccttccccaacacagccggtcactcagcggaccgcacacccatccttgagtctcctttgcagccctcagaactccagccccactacgtggccagccatccagagcccccagccggcttcgaagggcttcaggcggaggagtgcggcatcctgaacggctgtgagaatggccgctgtgtgcgcgtgcgggagggctacacctgtgactgttttgagggcttccagctggatgcggcccacatggcctgcgtagatgtgaatgagtgtgatgacttgaacgggcctgctgtgctctgtgtccatggttactgcgagaacacagagggctcctaccgctgccactgctccccgggatatgtggctgaggcagggcccccccactgcactgccaaggagtagcagtcaggggtcagtgtggcaactacctggaaatggcctccagtcacaggcaggggccttgaggatgatttcctagctgggaagacaccgtgacatcaggccagaggtttccaatcagccttgcctgctttcatctctcccagcttagcctctggctgtaagcttcggtcattgcctccatgcccttgcttggctcaagcaccaccaatcgctttaatgcttcagccaccgcatgaggccctgtccaccacctttcctggccttgctatgggatgcttaccaaaggatggccctcatccaccctcccaagctgtgcgagcatgcaaggccccatggcctcacactgcagacacccctttccagccacaatccaccatcatcctgacgatcccacaactgggacagaggctacatctgccctagggaggtccttcagaatctgtggagcaagaaaggatttggggaagcttggggactgactccagagccccctcctaagaaccatcaccaccactcagccaatctgttctgggccctgattttgccacacctccatcctgtagcccattctctgaccccaaggagtggcagaagatcccttcactcagagaagcaaggctgatattagcttgttgaatgtaagagacacaaatgaagaagaacaaagagcctgagaaagcagcaagaggacatgatgaaaaatacgtggagttgatgagaaaggggagccaaggctttatacgtctaaagaaaatattcagtagctgaatccgcccagtgatagcctgtgggcaccagcagcaagggctgccatgggatacagcacccatctacaaagacctctattacataaacactgcttcttacaggaaacaaacctcttctgggatctccttttgtgaaaaccagtttgatgtgctaaaagtaaaaagtctattttccagtgtggtcttgttcagaagcagccagatttccaatgttgtttttcccctccactcagaaacccctgccctttcccttcagaaaacgatggcaggcattcctctgagtttacaagcagagactcactccaacccaaactagctgggagttcagaaccatggtggaataaagaaatgtgcatctggtctcttctgttgtttttatttcatatcagattaaatttctttaccatgttggctaagtctaaatattagagatgaggctgtgcctactccctggccagctctgctgatagcctatgatgggttccaatgggaaatgactctttactattaaaagacaaggaaagctctgacttcgtacttctctgatgaatggcaatgtaaatgaacaaggctccatgtgactggagcatggaagtgaatgctactttcttaatttaatctgccctgtcctacctgctcctctgattgttagccatcacataacttattgaatgcttgccatgtgccaggcactgtgctgagtgccatacatacatttcatttaattatccaataatcctacttactattgtttattctcaatttacaggtgaggaaactgagacatagaaagcttaaataatttgctctaggtccctatactaattgagatgttttcagcagacaaaaataagtcactcttacgcatgtaaaaatattctttcactcataataaaagaaatgcaacttaaaaccatactgagatcctttttaaaactgtcagattggcaaagatcaaaaagcttgttaagactcctagctggcaagcatgtgggaaataggtactctcataacagctgacagaagtacatattgaagaggacaatatggcaatgtccttcaaaattataaaagcactaactctttgatccagtagtttccacttctaggaacttaaccttcagatatattttcaccagatataaaatgacatacgtaagaagttattactgcatcatttgtaatagctaaagattagaaacaattaattgctatggaacccttaaaaaaggtggctctaaattccttaagaaagatatccaaaacaaattaaggggaaaagggagggttgcataacagcatgtaacataacactatttgtctaaagaatggggaaagagaagcggttgtctgttcacctcatccttgccactccgccttatacctcaccatcggagaacttaccatcccccaaatatatcctgctggcactacaccttcgtaactgctgttcccactgcccccacaacactccaatctccaccttcctgccaaccttgagatattgtatgcatatatacatatttcctttgtttaaaatacaaatagcatactatgcatgctgttctgcactttgctttttttttatacttgcaattagagctgcctcatttcattttacagctgtatactattccattgaatggataaaccataatttatttaatcagtccctcactgacagacgtttccattgtttcctgtcttttgctataaataatgctgtaatgaatattcttgtgcatatttcacattaatttatgggattacatctgcaggataaattcttagaaccaatggtggtttgtggggagggtgacagggtggataggtacaggattgggagacttttcactgtatctccttttgtatttttttgaattttgtaccatatgtatcacctattcaaaaaaaataaaatatttttaaataca

SEQ ID NO: 10 - protein, 1821 aa, Homo sapiens
MRPRTKARSPGRALRNPWRGFLPLTLALFVGAGHAQRDPVGRYEPAGGDANRLRRPGGSYPAAAAAKVYSLFREQDAPVAGLQPVERAQPGWGSPRRPTEAEARRPSRAQQSRRVQPPAQTRRSTPLGQQQPAPRTRAAPALPRLGTPQRSGAAPPTPPRGRLTGRNVCGGQCCPGWTTANSTNHCIKPVCEPPCQNRGSCSRPQLCVCRSGFRGARCEEVIPDEEFDPQNSRLAPRRWAERSPNLRRSSAAGEGTLARAQPPAPQSPPAPQSPPAGTLSGLSQTHPSQQHVGLSRTVRLHPTATASSQLSSNALPPGPGLEQRDGTQQAVPLEHPSSPWGLNLTEKIKKIKIVFTPTICKQTCARGHCANSCERGDTTTLYSQGGHGHDPKSGFRIYFCQIPCLNGGRCIGRDECWCPANSTGKFCHLPIPQPDREPPGRGSRPRALLEAPLKQSTFTLPLSNQLASVNPSLVKVHIHHPPEASVQIHQVAQVRGGVEEALVENSVETRPPPWLPASPGHSLWDSNNIPARSGEPPRPLPPAAPRPRGLLGRCYLNTVNGQCANPLLELTTQEDCCGSVGAFWGVTLCAPCPPRPASPVIENGQLECPQGYKRLNLTHCQDINECLTLGLCKDAECVNTRGSYLCTCRPGLMLDPSRSRCVSDKAISMLQGLCYRSLGPGTCTLPLAQRITKQICCCSRVGKAWGSECEKCPLPGTEAFREICPAGHGYTYASSDIRLSMRKAEEEELARPPREQGQRSSGALPGPAERQPLRVVTDTWLEAGTIPDKGDSQAGQVTTSVTHAPAWVTGNATTPPMPEQGIAEIQEEQVTPSTDVLVTLSTPGIDRCAAGATNVCGPGTCVNLPDGYRCVCSPGYQLHPSQAYCTDDNECLRDPCKGKGRCINRVGSYSCFCYPGYTLATSGATQECQDINECEQPGVCSGGQCTNTEGSYHCECDQGYIMVRKGHCQDINECRHPGTCPDGRCVNSPGSYTCLACEEGYRGQSGSCVDVNECLTPGVCAHGKCTNLEGSFRCSCEQGYEVTSDEKGCQDVDECASRASCPTGLCLNTEGSFACSACENGYWVNEDGTACEDLDECAFPGVCPSGVCTNTAGSFSCKDCDGGYRPSPLGDSCEDVDECEDPQSSCLGGECKNTVGSYQCLCPQGFQLANGTVCEDVNECMGEEHCAPHGECLNSHGSFFCLCAPGFVSAEGGTSCQDVDECATTDPCVGGHCVNTEGSFNCLCETGFQPSPESGECVDIDECEDYGDPVCGTWKCENSPGSYRCVLGCQPGFHMAPNGDCIDIDECANDTMCGSHGFCDNTDGSFRCLCDQGFEISPSGWDCVDVNECELMLAVCGAALCENVEGSFLCLCASDLEEYDAQEGHCRPRGAGGQSMSEAPTGDHAPAPTRMDCYSGQKGHAPCSSVLGRNTTQAECCCTQGASWGDACDLCPSEDSAEFSEICPSGKGYIPVEGAWTFGQTMYTDADECVIFGPGLCPNGRCLNTVPGYVCLCNPGFHYDASHKKCEDHDECQDLACENGECVNTEGSFHCFCSPPLTLDLSQQRCMNSTSSTEDLPDHDIHMDICWKKVTNDVCSEPLRGHRTTYTECCCQDGEAWSQQCALCPPRSSEVYAQLCNVARIEAEREAGVHFRPGYEYGPGPDDLHYSIYGPDGAPFYNYLGPEDTVPEPAFPNTAGHSADRTPILESPLQPSELQPHYVASHPEPPAGFEGLQAEECGILNGCENGRCVRVREGYTCDCFEGFQLDAAHMACVDVNECDDLNGPAVLCVHGYCENTEGSYRCHCSPGYVAEAGPPHCTAKE

SEQ ID NO: 11 - DNA, 21 bp, artificial sequence
  synthetic nucleotide
ggaagtctagtgaccagaata

SEQ ID NO: 12 - DNA, 21 bp, artificial sequence
  synthetic nucleotide
gctggtgaaggtgcaaattca

SEQ ID NO: 13 - DNA, 21 bp, artificial sequence
  synthetic nucleotide
gcttctatgtggcgccaaatg

SEQ ID NO: 14 - DNA, 21 bp, artificial sequence
  synthetic nucleotide
gcaccaaccactgtatcaaac

SEQ ID NO: 15 - DNA, 20 bp, artificial sequence
  synthetic oligonucleotide primer
ctccaagcagatgcagcaga

SEQ ID NO: 16 - DNA, 23 bp, artificial sequence
  synthetic oligonucleotide primer
accatgatgatgcgcaaggccat

SEQ ID NO: 17 - DNA, 25 bp, artificial sequence
  synthetic oligonucleotide probe
ccgtggtgctgatggggggcaagaa

(19) United States
(12) Patent Application Publication
(10) Pub. No.: US 2023/0265427 A1
(43) Pub. Date: Aug. 24, 2023

(54) Treatment of Genetic Dilated Cardiomyopathies

(71) Applicants: Genethon, Evry-Courcouronnes (GR); Institut National de la Santé et de la Recherche Médicale, Paris (FR); Universite d'Evry Val d'Essonne, Evry (FR)

(72) Inventors: Isabelle RICHARD, Corbeil-Essonnes (FR); Ariane BIQUAND, Maisons Alfort (FR)

(73) Assignees: Genethon, Evry-Courcouronnes (FR); Institut National de la Santé et de la Recherche Médicale, Paris (FR); Universite d'Evry Val d'Essonne, Evry (FR)

(21) Appl. No.: 18/009,006

(22) PCT Filed: Jun. 9, 2021
(86) PCT No.: PCT/EP2021/065424

§ 371 Date: Dec. 8, 2022

(30) Foreign Application Priority Data
Jun. 9, 2020 (EP) 20305625.4

(51) Int. Cl. C12N 15/113; A61P 9/00; C12N 15/86

(52) U.S. Cl. CPC C12N 15/113; A61P 9/00; C12N 15/86; C12N 2310/13; C12N 2310/16; C12N 2310/14; C12N 2750/14141

(57) ABSTRACT

The invention relates to the treatment of genetic dilated cardiomyopathies using expressible modulators of the Wnt pathway or TGF-β pathway, preferably using gene transfer.

### FIELD OF THE INVENTION

The invention relates to the treatment of genetic dilated cardiomyopathies, using expressible modulators of the WNT pathway or TGF-β pathway, preferably using gene transfer.

### BACKGROUND OF THE INVENTION

Dilated cardiomyopathies (DCM or CMD) are characterized by hypokinesis of the myocardium and dilatation of the cardiac cavities. The cardiac remodelling that takes place during dilated cardiomyopathies consists of damage to the cardiomyocytes associated with the presence of fibrosis, which are inseparable from each other. The damage to the cardiomyocytes involves a decrease in their contractile capacity and a change in their structure, which leads to apoptosis and to the expansion of fibrosis, which replaces the necrotic cardiomyocytes. The proliferation of fibroblasts prevents compensatory hypertrophy of the cardiomyocytes. These manifestations will clinically translate into a decrease in cardiac function. This serious complication can be a cause of death.

A heritable pattern is present in 20 to 30% of DCM cases. Most familial DCM pedigrees show an autosomal dominant pattern of inheritance, usually presenting in the second or third decade of life (summary by Levitas et al., Europ. J. Hum. Genet., 2010, 18: 1160-1165). In Duchenne muscular dystrophy (DMD), a muscle disease due to mutation in the dystrophin gene, a dilated cardiomyopathy is clinically revealed around the age of 15 years and affects almost all patients after the age of 20 years. In the case of Becker muscular dystrophy (BMD), an allelic form of DMD, cardiac damage develops at the age of 20 years, and 70% of patients are affected after the age of 35. DCM due to titin, a giant protein of the sarcomere, is implicated in 1/250 cases of heart failure (Burke et al., JCI Insight. 2016; 1(6):e86898).

In genetically-induced dilated cardiomyopathies, most of the genes involved code for structural elements of cardiomyocytes, including extracellular matrix or Golgi apparatus proteins (laminin, fukutin) involved in cellular adhesion and signaling pathways; desmosome proteins (desmocollin, plakoglobin) involved in cellular junctions; sarcoplasmic reticulum proteins (RYR2, SERCA2a (ATP2A2), phospholamban) involved in calcium homeostasis; nuclear envelop proteins (lamin A/C) involved in myocardial structural organisation; cytoskeleton proteins (dystrophin, telethonin, α-actinin, desmin, sarcoglycans) involved in cytoskeleton integrity and muscular strength transmission; and sarcomer proteins (titin, troponin, myosin, actin) involved in generation and transmission of muscular strength

Therapeutic approaches are those used for the treatment of acquired dilated cardiomyopathies that are also valid for the management of genetic dilated cardiomyopathies such as for example in cases of DMD and titinopathies. There is currently no curative treatment for these pathologies. The drugs currently available for the treatment of acquired dilated cardiomyopathies will improve the symptoms but not treat the cause of the disease. The treatments prescribed are those for heart failure, accompanied by hygienic and dietetic measures such as reducing alcohol consumption, reducing water and salt intake and moderate and regular physical exercise. Among pharmaceutical treatments, angiotensin II converting enzyme inhibitors (ACE inhibitors) prevent the production of angiotensin II in order to decrease vasoconstriction and blood pressure. Diuretic drugs remove excess salt and water from the body by inhibiting renal sodium reabsorption. β-blockers, or β-adrenergic receptor antagonists, block the effects of adrenergic system mediators stimulated during dilated cardiomyopathies and decrease heart rate. Mineral-corticoid receptor antagonists block the binding of aldosterone and lower blood pressure. When rhythm disturbances are severe, anti-arrhythmic drugs such as amiodarone are prescribed. Implantation of a pacemaker and/or automatic defibrillator may also be considered. In the most severe cases, patients may benefit from a heart transplant (Ponikowski et al., European Heart Journal, 2016, 37, 2129-2200).

Corticosteroid treatment, frequently prescribed in DMD, allows an improvement of the muscular phenotype in the medium term thanks to a reduction in inflammation, but its action on the cardiac phenotype is subject to debate. The management of DMD related to dystrophin and titin requires an annual and systematic cardiac check-up (electrocardiogram and ultrasound). In particular, Perindopril, an angiotensin-converting enzyme inhibitor, has been shown to reduce mortality in DMD patients when taken as a preventive treatment from childhood onwards (Duboc et al., Journal of the American College of Cardiology, 2005, 45, 855-857). Molecules being tested in the treatment of cardiac impairment in DMD are mainly molecules already used in the treatment of heart failure. Other therapies are aimed at treating muscle and heart damage by reducing fibrosis. This is the case for Pamrevlumab (Phase II trial NCT02606136), a monoclonal antibody directed against connective tissue growth factor, and Tamoxifen (Phase I trial NCT02835079 and Phase III trial NCT03354039), an anti-estrogen.

There is therefore a medical need to develop new therapeutic strategies for genetic dilated cardiomyopathies.

The WNT (or Wnt) pathway orchestrates various biological processes, such as cell proliferation, differentiation, organogenesis, tissue regeneration and tumorigenesis. Classically, Wnt signaling is divided into β-catenin dependent (canonical, Wnt/β-catenin pathway) and β-catenin independent (noncanonical, Wnt/planar cell polarity (PCO) and calcium pathway). All three are activated by the binding of different secreted glycoproteins (Wnt ligands) to the frizzled (FZD) receptor family, to transduce a signaling cascade from the dishevelled protein (DVL) in the cell. The Wnt pathway is modulated by endogenous antagonists such as dickkopf (DKK3), Wnt inhibitory signaling protein, secreted frizzled related protein and cerberus. The secretion of WNT proteins mainly depends on acylation by porcupine (PORCN). β-catenin is a crucial signaling transducer in Wnt signaling. The 3-catenin protein destruction complex composed of adenomatous polyposis coli (APC), casein kinase 1 (CK1) and glycogen synthase kinase 3α/β (GSK-3α/β) and axin tightly controls β-catenin via phosphorylation-mediated proteolysis. The poly-ADP-ribosylating enzyme tankyrase interacts with and degrades axin via ubiquitin-mediated proteasomal degradation. In the absence of a ligand, the β-catenin accumulated in the cytoplasm is degraded by the destruction complex. Upon binding of one of the secreted Wnt proteins to its frizzled receptor (FZD) and lipoprotein co-receptor (LRP5/6), the cytoplasmic β-catenin is stabilized and translocated into the nucleus where it interacts with the transcription factor TCF/LEF and CBP to regulate its target genes (Rao et al., Circ. Res., 2010, 106, 1798-1806; The Wnt Homepage (http://www.stanford.edu/group/nusselab/cgi-bin/wnt/).

The WISP2/CCN5 or WNT1-inducible signaling pathway protein 2 is an activator of the WNT canonical pathway and a member of the NCC family of extracellular matrix proteins. The WISP2/CCN5 protein has the opposite effect of the connective tissue growth factor CTGF/CCN2: CCN2 acts as a cofactor of TGF-β in the induction of fibrogenesis, while WISP2/CCN5 inhibits cardiac fibrosis by inhibiting TGF-β signaling and fibroblast differentiation (Yoon et al., Journal of molecular and cellular cardiology, 2010, 49, 294-303).

DKK3 is a secreted protein present in developing and adult hearts and an antagonist of the WNT pathway.

SFRP2 is a secreted protein that binds to extracellular Wnt ligands and frizzled receptors, thus modulating the signaling cascade. SFRP2 is mainly an antagonist of the WNT pathway but it can also increase its signalling, thus SFRP2 has a major role in cardiac fibrosis, but its effect is still controversial: some studies describe it as pro-fibrotic or, on the contrary, anti-fibrotic (He et al., PNAS, 2010, 107, 21110-21115; Kobayashi et al. Nature Cell Biology, 2009, 11, 46-55; Lin et al., American Journal of physiology Cell physiology, 2016, 311, C710-C719; Mastri et al., American Journal of physiology Cell physiology, 2014, 306, C531-C539). A low concentration of SFRP2 may enhance the effects of the Wnt pathway and promote myocardial fibrosis, while a high concentration of SFRP2 may antagonize the Wnt pathway and inhibit myocardial fibrosis (Wu et al., International Journal of biological sciences, 2020, 16, 730-738). Its action in cardiac fibroblasts also appears to be implicated in TGF-related fibrosis-β1 (Ge and Greenspan, The journal of Cell Biology, 2006, 175, 111-120).

Aberrant upregulation of Wnt signaling is associated with cancer, osteoarthritis, and polycystic kidney disease, while aberrant downregulation of Wnt signaling has been linked to osteoporosis, diabetes, and neuronal degenerative diseases. The Wnt/β-catenin pathway is a therapeutic target for human cancers and various Wnt inhibitors have been examined in preclinical and clinical studies of various human cancers: PORCN inhibitors, Wnt ligand antagonists (FZD decoy receptor), FZD antagonists/monoclonal antibodies, CBP/β-catenin binding inhibitors and β-catenin target inhibitors. Tankyrase inhibitors, including XAV939, JW-55, RK-287107, and G007-LK, which downregulate 0-catenin stabilization by stabilizing axin, have also been developed (Review in Jung and Park, Experimental & Molecular Medicine, 2020, 52, 183-191; The Wnt Homepage (http://www.stanford.edu/group/nusselab/cgi-bin/wnt/).

The cytokines TGF-β are involved in many cellular functions, such as inflammation, cell proliferation and differentiation. The TGF-β pathway is composed of three cytokines of similar structure: TGF-01, 2 and 3 and transmembrane receptors. It mainly activates the Smad channel, but also the Erk, JNK, p38 MAPK and GTPase channels (Umbarkar et al., JACC Basic Transl. Sci., 2019, 4, 41-53). Aberrant upregulation of TGF-β signaling is associated with cancer and fibrosis. The TGF-β pathway is a therapeutic target for human cancers and fibrotic diseases. TGF-β pathway inhibitors have been examined in preclinical and clinical studies of various human cancers and fibrotic diseases such as idiopathic pulmonary fibrosis; scleroderma, scarring and others: anti-TGFβ2 antisense oligonucleotides (AP-12009, AP-11014, NovaRx); small-molecule TGFβRI or TGFβRI&RII kinase inhibitors (LY-2157299, SB-431542 and many others); anti-pan TGFβ antibody (GC-1008; ID11, SR-2F, 2G7); peptide-fragment of TGFβRIII (P-144); Smad-interacting peptide aptamers (Trx-xFoxHlb/Trx-Lefl); anti-TGF02 antibody (Lerdelimumab or CAT-152); anti-TGFβ1 antibody (Metelimumab or CAT-192); stabilized soluble TGFβRII (soluble TBR2-Fc); Review in Akhurst, R J, Current Opinion in Investigational Drugs, 2006, 7, 513-521; Nagaraj N. S. & Datta P. K., Expert Opin. Investig Drugs, 2010, 19, 77-91).

Cartilage Intermediate Layer Protein 1 (CILP-1) is a matri-cellular protein found mainly in the chondrocytes of articular cartilage, but its expression has recently been found to be significantly higher in human idiopathic dilated cardiomyopathy and infarction (van Nieuwenhoven et al., Scientific Reports, 2017, 7, 16042; Yung et al., Genomics, 2004, 83, 281-297). In the hearts of normal mice, CILP is expressed by cardiomyocytes and fibroblasts, and the protein is found in the cytosol, nuclear fraction and extracellular matrix (van Nieuwenhoven et al., Scientific Reports, 2017, 7, 16042; Zhang et al., Journal of molecular and cellular cardiology, 2018, 116, 135-144). The expression of CILP-1 protein is increased in a mouse model of induced cardiac fibrosis and its expression is stimulated by TGF-01 (Mori et al., Biochemical and biophysical research communications, 2006, 341, 121-127).

The LTBP-2 protein is a protein of the Latent TGFβ1-Binding Protein family, extracellular matrix proteins related to the TGF-β pathway. LTBP2 regulates the release of TGF-β1 and TGF-β1 promotes the expression of LTBP-2 (Bai et al., Biomarkers, 2012, 17, 407-415; Sinha et al., Cardiovascular Research, 2002, 53, 971-983). In addition, LTBP-2 is highly expressed and localized in the fibrotic regions of the myocardium in mice and patients after cardiac arrest (Gabrielsen et al., Journal of molecular and cellular cardiology, 2007, 42, 870-883; Park et al., Circulation, 2018, 138, 1224-1235).

Wnt and TGF-β signaling are also targets for regenerative medicine through differentiation of mammalian pluripotent stem cell population or reprogramming of mammalian differentiated cells.

### SUMMARY OF THE INVENTION

The inventors have found that the Wnt and TGF-β pathways are both dysregulated and their genes overexpressed in two models of genetically-induced dilated cardiomyopathies, Duchenne muscular dystrophies (DBA2mdx mice) and titinopathies (DeltaMex5 mice). The most overexpressed genes included the WISP2, DKK3 and SFRP2 genes of the Wnt pathway and the CILP-1 and LTBP-2 genes of the TGF-β pathways. Using the DeltaMex5 mice model which is a severe model of genetic dilated cardiomyopathies, the inventors have shown that gene transfer-mediated modulation of the Wnt pathway by overexpression of WISP2, DKK3 or SFRP2 and inhibition of the TGF-β pathway by overexpression of CILP-1 or inhibition of LTBP-2 in DeltaMex5 mice showed significant improvement in cardiac fibrosis.

These results indicate that modulation of the Wnt pathway, in particular by overexpression of WISP2, DKK3 or SFRP2 and modulation of the TGF-β pathway by overexpression of CILP-1 or inhibition of LTBP-2 represent a therapeutic approach for genetically-induced cardiomyopathy such as titinopathy for which gene transfer approaches are not possible because of the size of the gene.

The present invention relates to an expressible modulator of the Wnt or TGF-β pathway for use in the treatment of genetic dilated cardiomyopathies.

In some embodiments, the modulator modulates the activity of a target protein of the Wnt or TGF-β pathway and is selected from the group consisting of: aptamer; antibody, recombinant target protein, inhibitory peptide, fusion protein, decoy receptor, soluble protein and dominant negative mutant.

In some embodiments, the modulator modulates the expression of a target gene of the Wnt or TGF-β pathway and is selected from the group consisting of: interfering RNA molecule, ribozyme, genome or epigenome editing enzyme complex, and target transgene.

In some embodiments, the modulator is an inhibitor or activator of the Wnt pathway or an inhibitor of the TGF-β pathway.

In some preferred embodiments, the modulator is an activator of CILP-1, CCN5/WISP2, DKK3 or SFRP2, or an inhibitor of LTBP2.

In some more preferred embodiments, the modulator is an interfering RNA which specifically decreases LTBP2 expression; preferably a shRNA comprising at least one sequence selected from the group consisting of SEQ ID NO: 11 to 14.

In some more preferred embodiments, the modulator is a transgene encoding CILP-1, DKK3, SRFP2, or CCN5/WISP2 protein or a variant thereof. Preferably, the CILP-1, DKK3, SRFP2, CCN5/WISP2 protein or a variant thereof comprises a sequence selected from the group consisting of the sequences SEQ ID NO: 2, 4, 6 and 8 and the sequences having at least 85% identity with any one of said sequences.

In some preferred embodiments, the modulator is inserted into a nucleic acid construct comprising a cardiac promoter selected from the group consisting of: human cardiac troponin T promoter (TNNT2), alpha myosin heavy chain promoter (α-MHC), the myosin light chain 2v promoter (MLC-2v), the myosin light chain 2a promoter (MLC-2a), the CARP gene promoter, the alpha-cardiac actin promoter, the alpha-tropomyosin promoter, the cardiac troponin C promoter, the cardiac myosin-binding protein C promoter, and the sarco/endoplasmic reticulum Ca²⁺ ATPase (SERCA) promoter; preferably, the human cardiac troponin T promoter. t.

In some more preferred embodiments, the nucleic acid construct is contained in a vector for gene therapy; said vector advantageously comprises a viral particle, preferably an adeno-associated viral (AAV) particle. Sait AAV particle preferably comprises capsid protein(s) derived from AAV serotypes selected from the group consisting of: AAV-1, AAV-6, AAV-8, AAV-9 and AAV9.rh74 serotypes; more preferably AAV9.rh74.

In some more preferred embodiments, the genetic cardiomyopathy is caused by mutation in a gene selected from the group consisting of: laminin, emerin, fukutin, fukutin-related protein, desmocollin, plakoglobin, ryanodine receptor 2, sarcoplasmic reticulum Ca(2+) ATPase 2 isoform alpha, phospholamban, lamin A/C, dystrophin, telethonin, actinin, desmin, sarcoglycans, titin, cardiac troponin, myosin, cardiac actin, RNA binding motif protein 20, BCL2-associated athanogene 3, desmoplakin, tafazzin and sodium channels; preferably in the dystrophin or titin gene.

### DETAILED DESCRIPTION OF THE INVENTION

### Modulators

The invention relates to the use of an expressible modulator of the Wnt or TGF-β pathway for the treatment of genetic dilated cardiomyopathies (DCM).

As used herein, “a modulator” refers to an activator or inhibitor”

As used herein “an expressible modulator» refers to a modulator (activator or inhibitor) which can be produced by recombinant DNA technologies or delivered by gene transfer. Therefore, an expressible modulator consists of a ribonucleic acid (RNA) molecule or a protein, polypeptide or peptide. The invention encompasses in particular RNA molecule inhibitors such as interfering RNAs (siRNA, shRNA), CRISPR guide RNAs, ribozymes and aptamers targeting components of the Wnt or TGF-β pathway. The invention encompasses also protein, polypeptide or peptide modulators such as components of the Wnt or TGF-β pathway, variants or derivative thereof (fragments, fusion proteins; decoy receptors, soluble proteins, dominant negative mutants) and antibodies directed to components of the Wnt or TGF-β pathway including fragments and expressible derivative thereof.

As used herein peptide or polypeptide are used interchangeably to refer to a peptide or protein fragment of any length.

As used herein, the term “cardiac cells” includes in particular cardiac myocytes, myoblasts and stem-cells.

“a”, “an”, and “the” include plural referents, unless the context clearly indicates otherwise. As such, the term “a” (or “an”), “one or more” or “at least one” can be used interchangeably herein; unless specified otherwise, “or” means “and/or”.

As used herein, “a component of the Wnt or TGF-β (TGF-beta) pathway” refers to any component of this pathway including a ligand, receptor, signaling molecule, or modulator (activator or modulator) of the Wnt or TGF-β pathway. Such components are well-known in the art (see for example, Rao et al., Circ. Res., 2010, 106, 1798-1806 and Umbarkar et al., JACC Basic Transl. Sci., 2019, 4, 41-53; The Wnt Homepage (http://www.stanford.edu/group/nusselab/cgi-bin/wnt/).

A “modulator of the Wnt or TGF-β pathway”, “modulator of Wnt or TGF-β signaling” or “modulator of the Wnt or TGF-β signaling pathway” refers to a compound or molecule which activates or inhibits Wnt or TGF-β signaling, e.g., the transcription of Wnt or TGF-β target gene(s) through the signaling cascade transduced by the binding of a Wnt ligand or TGF-β cytokine to its cognate receptor. The modulator acts on a specific component of the Wnt or TGF-β pathway (Wnt or TGF-β pathway target gene or protein).

The modulator may inhibit or activate the expression or activity of a component of the pathway. The target may be any component of the Wnt or TGF-β pathway such as a ligand, receptor, signaling molecule, or modulator of the Wnt or TGF-β pathway. An activator may activate the pathway directly or inhibit the expression or activity of an inhibitor. Likewise an inhibitor may inhibit the pathway directly or activate the expression or activity of an inhibitor. The modulation may be direct or indirect. A direct modulation is directed specifically to the target. An indirect modulation is directed to any co-effector of the target such as with no limitations: a ligand or co-ligand, a receptor or co-receptor, or a co-factor of said target. The modulator (inhibitor or activator) may bind to a specific target protein of the Wnt or TGF-β pathway and disrupt or promote specific protein/protein interactions of the target or modulate the activity or function of the target. Alternatively, the modulator inhibits or activates the expression of a target gene of the Wnt or TGF-β pathway. The modulator may be an inhibitor which binds to a specific sequence of a target gene transcript (mRNA) and inhibits expression of the target gene. The modulator may be a transgene of the target gene which produces the surexpression of the target gene and protein or a recombinant target protein which increases the activity of the target protein.

Typically a modulator of the Wnt or TGF-β pathway refers to a compound that modulates Wnt or TGF-β signaling by at least 20%, 30%, 40%, 50%, 60% and preferably more than 70%, even more preferably more than 80%, more than 90%, more than 95%, more than 99% or even 100% (corresponding to no detectable activity) in a subject (or in a cell in vitro) as compared to the Wnt or TGF-β signaling prior to or in the absence or, administration of said compound.

A modulator of the Wnt or TGF-β pathway can be identified by various assays that are well-known in the art such as cellular Wnt or TGF-β reporter assays. Examples of Wnt reporter assay include the TOP-flash assay (Molenaar et al., Cell, 1996, 86, 391-399) which is widely used and variants of TOP-flash are available. Another assay is the TCF/LEF-reporter assay using SW480 cells bearing a mutation in the APC protein which leads to constitutively active canonical Wnt signaling (Deshmukh et al., Osteoarthritis and Cartilage, 2018, 26, 18-27). Examples of TGF-β reporter assay include TGF-β/SMAD luciferase reporter cell lines or lentivirus vectors.

Typically a modulator of the Wnt or TGF-β pathway may be a compound that modulates (increases or decreases) expression or activity of a component of the Wnt or TGF-β pathway (Wnt or TGF-β target gene or protein) by at least 20%, 30%, 40%, 50%, 60% and preferably more than 70%, even more preferably more than 80%, more than 90%, more than 95%, more than 99% or even 100% (corresponding to no detectable activity) in a subject (or in a cell in vitro) as compared to the expression or activity of the component of the Wnt or TGF-β pathway prior to or in the absence or, administration of said compound. As used herein, modulation of Wnt or TGF-β pathway target gene expression includes any increase or decrease in expression or protein activity or level of the Wnt or TGF-β pathway target gene or protein encoded by said Wnt or TGF-β pathway target gene as compared to a situation wherein no modulation has been induced. The increase or decrease can be of at least, 20%, 30%, 40%, 50%, 60%, 70%, 80%, 90%, 95%, 99% as compared to the expression of Wnt or TGF-β pathway target gene or level of the Wnt or TGF-β pathway target protein which has not been targeted by modulation.

The expression level of Wnt or TGF-β target gene transcript (mRNA) may be determined by any suitable methods known by skilled persons. For example the nucleic acid contained in the sample is first extracted according to standard methods, for example using lytic enzymes or chemical solutions or extracted by nucleic-acid-binding resins following the manufacturer's instructions. The extracted mRNA is then detected by hybridization (e.g., Northern blot analysis) and/or amplification (e.g., RT-PCR). The expression level of target protein may also be determined by any suitable methods known by skilled persons. The quantity of the protein may be measured, for example, by semi-quantitative Western blots, enzyme-labelled and mediated immunoassays, such as ELISAs, biotin/avidin type assays, radioimmunoassay, immunoelectrophoresis, mass spectrometry, or immunoprecipitation or by protein or antibody arrays.

In the context of the present invention, a modulator of the Wnt or TGF-β pathway according to the present invention is preferably selective for its target protein or gene. By “selective” it is meant that the affinity of the modulator is at least 10-fold, preferably 25-fold, more preferably 100-fold, and still preferably 500-fold higher than the affinity for another protein or gene.

The Wnt or TGF-β pathway modulator is used to improve cardiac fibrosis in subjects suffering from genetic dilated cardiomyopathies (DCM). As shown in the examples of the present application, the Wnt or TGF-β pathway modulator is necessary and sufficient to improve fibrosis in subjects suffering from genetic dilated cardiomyopathies (DCM), in particular genetic cardiomyopathy. Improvement of fibrosis can be determined by administration of the Wnt or TGF-β pathway modulator in animal models of DCM such as mouse models that are well-known in the art and disclosed in the examples of the present application; DeltaMex5 mice which have the deletion of the penultimate exon (Mex5) of the titin gene (titin^{Mex5-/Mex5-}; Charton et al., Human molecular genetics, 2016, 25, 4518-4532) and DBA2mdx mice which have a punctual mutation on exon 23 of the dystrophin gene. Improvement of fibrosis in the heart may be determined by a decrease of fibrotic tissue in the heart after histological analysis (Sirius Red staining) or a decrease of fibrosis marker level (fibronectin, vimentin, collagen 1a1 and collagen 3a1) in the heart after RT-PCR or immunohistological analysis in the treated animals compared to untreated controls. Normal mice (untreated) are advantageously used as positive control to evaluate the efficiency of the treatment in the diseased mice.

According to the invention, a modulator of the Wnt or TGF-β pathway can be selected among any expressible compound having the ability to modulate gene expression of a Wnt or TGF-β pathway target gene or activity of a Wnt or TGF-β pathway target protein.

In some embodiments, the modulator modulates the activity of a target protein of the Wnt or TGF-β pathway. The modulator of activity may be selected from the group comprising: aptamers; antibodies (agonists and antagonists) directed to the target protein or its ligand, receptor, co-receptor, including antibody fragments and expressible derivative thereof; recombinant target protein, target protein variants or derivative thereof such as fusion proteins, soluble proteins and dominant negative mutants; decoy receptors and inhibitory peptides.

Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by Exponential enrichment (SELEX) of a random sequence library, as described in Tuerk C. and Gold L., 1990 and can be optionally chemically modified. Smad-interacting peptide aptamers (Trx-xFoxH1b/Trx-Lef1) are TGF-beta inhibitors (Review in Akhurst, R J, Current Opinion in Investigational Drugs, 2006, 7, 513-521; Nagaraj N. S. & Datta P. K., Expert Opin. Investig Drugs, 2010, 19, 77-91).

As used herein, the term “antibody” refers to a protein that includes at least one antigen-binding region of immunoglobulin. The antigen binding region may comprise one or two variable domains, such as for example a VH domain and a VL domain or a single VHH or VNAR domain. The term “antibody” encompasses full length immunoglobulins of any isotype, functional fragments thereof comprising at least the antigen-binding region and derivatives thereof. Antigen-binding fragments of antibodies include for example Fv, scFv, Fab, Fab′, F(ab′)2, Fd, Fabc and sdAb (V_{H}H, V-NAR). Antibody derivatives include with no limitation polyspecific or multivalent antibodies, intrabodies and immunoconjugates. Intrabodies are antibodies that bind intracellularly to their antigen after being produced in the same cell (for a review see for example, Marschall A L, Dubel S and Böldicke T “Specific in vivo knockdown of protein function by intrabodies”, MAbs. 2015; 7(6):1010-35). The antibody may be glycosylated. An antibody can be functional for antibody-dependent cytotoxicity and/or complement-mediated cytotoxicity, or may be non-functional for one or both of these activities. Antibodies are prepared by standard methods that are well-known in the art such as hybridoma technology, selected lymphocyte antibody method (SLAM), transgenic animals, recombinant antibody libraries or synthetic production. Several anti-TGF-β antibodies have been examined in preclinical and clinical trials of human cancer of fibrotic diseases; anti-pan TGFβ antibody (GC-1008; ID11, SR-2F, 2G7); anti-TGF02 antibody (Lerdelimumab or CAT-152); anti-TGFβ1 antibody (Metelimumab or CAT-192); Review in Akhurst, R J, Current Opinion in Investigational Drugs, 2006, 7, 513-521; Nagaraj N. S. & Datta P. K., Expert Opin. Investig Drugs, 2010, 19, 77-91. FZD antagonist monoclonal antibodies which bind to FZD receptor and block the bind od WNT ligands have been developed: Vantictumab (OMP-18R5); OTSA101 (Review in Jung and Park, Experimental & Molecular Medicine, 2020, 52, 183-191; The Wnt Homepage (http://www.stanford.edu/group/nusselab/cgi-bin/wnt/).

Other peptide inhibitors of the TGF-β pathway include the peptide-fragment of TGFβRIII (P-144) and the stabilized soluble TGFβRII (soluble TBR2-Fc); Review in Akhurst, R J, Current Opinion in Investigational Drugs, 2006, 7, 513-521; Nagaraj N. S. & Datta P. K., Expert Opin. Investig Drugs, 2010, 19, 77-91. Peptide inhibitors of the WNT pathway include: Dickkopf (Dkk), Axin, GSK, SFRP (Secreted frizzled-related proteins) and SRFP peptides; FZD decoy receptor: OMP-54F28 comprises the cysteine-rich domain of FZD8 fused to human Ig Fc domain; dominant negative Dishevelled or TCF. OTSA101 (Review in Jung and Park, Experimental & Molecular Medicine, 2020, 52, 183-191; The Wnt Homepage (http://www.stanford.edu/group/nusselab/c_{g}i-bin/wnt/).

In some embodiments, the modulator modulates the expression of a target gene of the Wnt or TGF-β pathway. The modulator of expression may be selected from the group comprising: interfering RNA molecules, ribozymes and genome or epigenome editing enzyme complexes, and target transgene. Interfering RNA molecules include with no limitations siRNA and shRNA. Genome and Epigenome editing system may be based on any known system such as CRISPR/Cas, TALENs, Zinc-Finger nucleases and meganucleases. Interfering RNA molecules, ribozymes, genome and epigenome editing enzymes are well-known in the art and inhibitors a target gene of the WNT or TGF-β pathway according to the invention may be easily designed based on these technologies using the sequences of the genes of the WNT or TGF-β pathway that are well-known in the art.

In some particular embodiments, the modulator is an activator or inhibitor of the Wnt pathway or an inhibitor of the TGF-β pathway according to the present disclosure. In some particular embodiments, the modulator is an activator of the Wnt pathway. In some particular embodiments, the modulator is an inhibitor of the Wnt or TGF-β pathway according to the present disclosure. In some particular embodiments, the modulator is an inhibitor of the Wnt pathway according to the present disclosure. In some particular embodiments, the modulator is an inhibitor of the TGF-β pathway according to the present disclosure.

In some embodiments, the modulator targets a gene or protein of the WNT or TGF-β pathway selected from the group consisting of: CILP-1 and LTBP2 from the TGF-β pathway; CCN5/WISP2, DKK3 and SFRP2 from the WNT pathway. In some preferred embodiments, the modulator according to the present disclosure is an activator of CILP-1, CCN5/WISP2, DKK3 or SFRP2, or an inhibitor of LTBP2.

The gene Cartilage intermediate layer protein (CILP or CILP-1) (Gene ID: 8483) encodes a CILP-1 preprotein (GenBank/NCBI accession number: NP_003604.4 accessed on 25 Apr. 2020; SEQ ID NO: 2). The mRNA has the sequence GenBank accession number NM_003613.4 accessed on 25 Apr. 2020; SEQ ID NO: 1). CLIP-1 preprotein has a 1184 amino acid sequence comprising a signal peptide (positions 1-21); a proprotein (positions 22-1184); a CILP-1 N-terminal domain (positions 22-720); CILP-1 C-terminal domain (positions 725-1184). The full length and the N-terminal domain function as an IGF-1 antagonist. Two CILP-1 isoforms X1 and X2 are disclosed (GenBank accession number XP_016878167.1 and XP_016878168.1 accessed on 28 May 2020).

The gene dickkopf WNT signaling pathway inhibitor 3 (DKK3) (GeneID:27122) encodes a DKK3 protein precursor (GenBank/NCBI accession number NP_056965.3 accessed on 27 Apr. 2020; SEQ ID NO: 4). The mRNA (transcript variant 1) has the sequence GenBank/NCBI accession number NM_015881.5 accessed on 27 Apr. 2020 (SEQ ID NO: 3). DKK3 protein precursor has a 350 amino acid comprising a signal peptide (positions 1-21). The mature protein is from positions 22-350.

The gene secreted frizzled related protein 2 (SFRP2) (GeneID: 6423) codes for a SRFP2 protein precursor (GenBank/NCBI accession number NP_003004.1 accessed on 31 May 2020; SEQ ID NO: 6). The mRNA has the sequence GenBank/NCBI accession number NM_003013.3 accessed on 31 May 2020 (SEQ ID NO: 5). SRFP2 protein precursor has a 295 amino acid sequence comprising a signal peptide (positions 1-19). The mature protein is from positions 20-295.

The gene cellular communication network factor 5 (CCN5), (GeneID: 8839) codes for a CCN5/WISP2 protein precursor (GenBank/NCBI accession number NP_003872.1 accessed on 3 May 2020; SEQ ID NO: 8). The mRNA (transcript variant 3) has the sequence GenBank/NCBI accession number NM_003881.3 accessed on 3 May 2020 (SEQ ID NO: 7). CCN5/WISP2 protein precursor has a 250 amino acid sequence comprising a signal peptide (positions 1-23). The mature protein is from positions 24-250.

The gene latent transforming growth factor beta binding protein 2 (LTBP2) (GeneID: 4053) codes for a LTBP2 protein precursor (GenBank/NCBI accession number NP_000419.1 accessed on 8 May 2020; SEQ ID NO: 10). The mRNA has the sequence GenBank/NCBI accession number NM_000428.3 accessed on 8 May 2020, mRNA; SEQ ID NO: 9). LTBP2 protein precursor has a 1821 amino acid sequence comprising a signal peptide (positions 1-35). The mature protein is from positions 36-1821.

The gene sequences of a number of different mammalian CILP-1, DKK3, SFRP2, CCN5 and LTBP2 proteins are known including, but being not limited to, human, pig, chimpanzee, dog, cow, mouse, rabbit or rat, and can be easily found in sequence databases.

### LTBP2 Inhibitor

In some preferred embodiment, the modulator is a LTBP2 inhibitor. In a particular embodiment, said LTBP2 inhibitor is an interfering RNA which specifically decreases, inhibits or represses LTBP2 expression.

As used herein, the term “iRNA”, “RNAi”, “interfering nucleic acid” or “interfering RNA” means any RNA which is capable of down-regulating the expression of the targeted protein. Nucleic acid molecule interference designates a phenomenon by which dsRNA specifically suppresses expression of a target gene at post-transcriptional level. In normal conditions, RNA interference is initiated by double-stranded RNA molecules (dsRNA) of several thousands of base pair length. In vivo, dsRNA introduced into a cell is cleaved into a mixture of short dsRNA molecules called siRNA. The enzyme that catalyzes the cleavage, Dicer, is an endo-RNase that contains RNase III domains (Bernstein, Caudy et al. 2001 Nature. 2001 Jan. 18; 409(6818):363-6). In mammalian cells, the siRNAs produced by Dicer are 21-23 bp in length, with a 19 or 20 nucleotides duplex sequence, two-nucleotide 3′ overhangs and 5′-triphosphate extremities (Zamore, Tuschl et al. Cell. 2000 Mar. 31; 101(1):25-33; Elbashir, Lendeckel et al. Genes Dev. 2001 Jan. 15; 15(2): 188-200; Elbashir, Martinez et al. EMBO J. 2001 Dec. 3; 20(23):6877-88).

Said interfering RNA can be as non-limiting examples small inhibitory RNAs (siRNAs) or short hairpin RNA.

In another embodiment, small inhibitory RNAs (siRNAs) is used to decrease the LTBP2 expression level in the present disclosure. LTBP2 gene expression can be reduced by administrating into a subject a small double stranded RNA (dsRNA), or a vector or construct causing the production of a small double stranded RNA, such that LTBP2expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

In a preferred embodiment, short hairpin RNA (shRNA) is used to decrease the CILP-1 expression level in the present disclosure. A short hairpin RNA (shRNA) is a sequence of RNA that makes a tight hairpin turn that can be used to silence target gene expression via RNA interference (RNAi). Expression of shRNA in cells is typically accomplished by delivery of plasmids or through viral or bacterial vectors. The promoter choice is essential to achieve robust shRNA expression. At first, polymerase III promoters such as U6 and HI were used; however, these promoters lack spatial and temporal control. As such, there has been a shift to using polymerase II promoters to regulate expression of shRNA.

Interfering nucleic acid are usually designed against a region 19-50 nucleotides downstream the translation initiator codon, whereas 5′UTR (untranslated region) and 3′UTR are usually avoided. The chosen interfering nucleic acid target sequence should be subjected to a BLAST search against EST database to ensure that the only desired gene is targeted. Various products are commercially available to aid in the preparation and use of interfering nucleic acid.

In a particular embodiment, the interfering nucleic acid is a siRNA of at least about 10-40 nucleotides in length, preferably about 15-30 base nucleotides. In particular, interfering nucleic acid according to the disclosure comprises at least one sequence selected from the group consisting of: 5′-GGAAGTCTAGTGACCAGAATA-3′ (SEQ ID NO: 11); 5′-GCTGGTGAAGGTGCAAATTCA-3′ (SEQ ID NO: 12); 5′-GCTTCTATGTGGCGCCAAATG-3′ (SEQ ID NO: 13); and 5′-GCACCAACCACTGTATCAAAC-3′ (SEQ ID NO: 14).

In a more preferred embodiment, up to four interfering nucleic acids comprising each a sequence SEQ ID NO: 11 to 14 are used concomitantly.

In a preferred embodiment, said interfering nucleic acid is a shRNA comprising at least one sequence selected from the group consisting of SEQ ID NO: 11 to 14, preferably comprising all the sequences SEQ ID NO: 11 to 14.

An interfering nucleic acid for use in the disclosure can be constructed using procedures known in the art. Particularly, interfering RNA can be produced by in vitro transcription from linear (e.g. PCR products) or circular templates (e.g., viral or non-viral vectors), or produced by in vivo transcription from viral or non-viral vectors. Interfering nucleic acid may be modified to have enhanced stability, nuclease resistance, target specificity and improved pharmacological properties. For example, antisense nucleic acid may include modified nucleotides or/and backbone designed to increase the physical stability of the duplex formed between the antisense and sense nucleic acids.

In another particular embodiment, LTBP2 inhibitor is a specific nuclease able to target and inactivate LTBP2 gene. Different types of nucleases can be used, such as Meganucleases, TAL-nucleases, zing-finger nucleases (ZFN), or RNA/DNA guided endonucleases like Cas9/CRISPR or Argonaute.

By “inactivating a target gene”, it is intended that the gene of interest is not or less expressed in a functional protein form. In particular embodiment, said nuclease specifically catalyzes cleavage in one targeted gene thereby inactivating said targeted gene.

The term “nuclease” refers to a wild type or variant enzyme capable of catalyzing the hydrolysis (cleavage) of bonds between nucleic acids within a DNA or RNA molecule, preferably a DNA molecule. In a particular embodiment, said nuclease according to the present disclosure is a RNA-guided endonuclease such as the Cas9/CRISPR complex. RNA guided endonucleases is a genome engineering tool where an endonuclease associates with a RNA molecule. In this system, the RNA molecule nucleotide sequence determines the target specificity and activates the endonuclease (Gasiunas, Barrangou et al. 2012; Jinek, Chylinski et al. 2012; Cong, Ran et al. 2013; Mali, Yang et al. 2013). Cas9/CRISPR involves a Cas9 nuclease and a guide RNA, also referred here as single guide RNA. Said single guide RNA is preferably able to target LTBP2 gene.

The inactivation of said target gene can also be performed by the use of site-specific base editors, for example by introducing premature stop codon(s), deleting a start codon or altering RNA splicing. Base editing directly generates precise point mutations in DNA without creating DNA double strand breaks. In a particular embodiment, base editing is performed by using DNA base editors which comprise fusions between a catalytically impaired Cas nuclease and a base modification enzyme that operates on single-stranded DNA (for review, see Rees H. A. et al. Nat Rev Genet. 2018. 19(12):770-788.

### CILP-1, DKK3, SRFP2, CCN5/WISP2 Activator

In other preferred embodiments, the modulator is a CILP-1, DKK3, SRFP2, CCN5/WISP2 activator.

In particular, the activator is a recombinant CILP-1, DKK3, SRFP2, or CCN5/WISP2 protein or a transgene encoding said protein.

In some preferred embodiments, the activator is a transgene encoding CILP-1, DKK3, SRFP2, or CCN5/WISP2 or a variant thereof.

As used herein, the term “transgene” refers to exogenous DNA or cDNA encoding a gene product. The gene product may be an RNA, peptide or protein. In addition to the coding region for the gene product, the transgene may include or be associated with one or more elements to facilitate or enhance expression, such as a promoter, enhancer(s), response element(s), reporter element(s), insulator element(s), polyadenylation signal(s) and/or other functional elements. Embodiments of the disclosure may utilize any known suitable promoter, enhancer(s), response element(s), reporter element(s), insulator element(s), polyadenylation signal(s) and/or other functional elements. Suitable elements and sequences will be well known to those skilled in the art.

The transgene according to the disclosure may be any nucleic acid sequence encoding a CILP-1, DKK3, SRFP2, or CCN5/WISP2 protein, in particular a native mammalian, preferably human, CILP-1, DKK3, SRFP2, or CCN5/WISP2 protein, or a variant thereof. Human, CILP-1, DKK3, SRFP2, and CCN5/WISP2 protein correspond to the sequences SEQ ID NO: 2, 4, 6 and 8, respectively. The coding sequences of a number of different mammalian CILP-1, DKK3, SRFP2, or CCN5/WISP2 proteins are known including, but being not limited to, human, pig, chimpanzee, dog, cow, mouse, rabbit or rat, and can be easily found in sequence databases. Alternatively, the coding sequence may be easily determined by the skilled person based on the polypeptide sequence.

In a preferred embodiment, said transgene comprises a coding sequence for CILP-1, DKK3, SRFP2, or CCN5/WISP2 protein which can be selected from the group consisting of the sequence SEQ ID NO: 1, 3, 5 and 7 and the sequences having at least 70%, 75%, 80%, 85%, 90%, or 95% identity with any one of said sequences.

In a particular embodiment, the transgene according to the disclosure may be any nucleic acid sequence encoding a CILP-1, DKK3, SRFP2, or CCN5/WISP2 protein variant.

As used herein, the term “variant” refers to a functional variant, e.g. which is capable of modulating the Wnt or TGF-β pathway.

Preferably, as used herein, the term “variant” refers to a polypeptide having an amino acid sequence having at least 70%, 75%, 80%, 85%, 90%, 95% or 99% identity to the native sequence. As used herein, the term “sequence identity” or “identity” refers to the number (%) of matches (identical amino acid residues) in positions from an alignment of two polypeptide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithms (e.g. Needleman and Wunsch algorithm; Needleman and Wunsch, 1970) which aligns the sequences optimally over the entire length, while sequences of substantially different lengths are preferably aligned using a local alignment algorithm (e.g. Smith and Waterman algorithm (Smith and Waterman, 1981) or Altschul algorithm (Altschul et al, 1997; Altschul et al., 2005). Alignment for purposes of determining percent amino acid sequence identity can be achieved in various ways that are within the skill in the art, for instance, using publicly available computer software available on internet web sites such as http://blast.ncbi.nlm.nih.gov/ or http://www.ebi.ac.uk/Tools/emboss/. Those skilled in the art can determine appropriate parameters for measuring alignment, including any algorithms needed to achieve maximal alignment over the full length of the sequences being compared. For purposes herein, % amino acid sequence identity values refers to values generated using the pair wise sequence alignment program EMBOSS Needle that creates an optimal global alignment of two sequences using the Needleman-Wunsch algorithm, wherein all search parameters are set to default values, i.e. Scoring matrix=BLOSUM62, Gap open=10, Gap extend=0.5, End gap penalty=false, End gap open=10 and End gap extend=0.5.

More preferably, the term “variant” refers to a polypeptide having an amino acid sequence that differs from a native sequence by less than 30, 25, 20, 15, 10 or 5 substitutions, insertions and/or deletions. In a preferred embodiment, the variant differs from the native sequence by one or more conservative substitutions, preferably by less than 15, 10 or 5 conservative substitutions. Examples of conservative substitutions are within the groups of basic amino acids (arginine, lysine and histidine), acidic amino acids (glutamic acid and aspartic acid), polar amino acids (glutamine and asparagine), hydrophobic amino acids (methionine, leucine, isoleucine and valine), aromatic amino acids (phenylalanine, tryptophan and tyrosine), and small amino acids (glycine, alanine, serine and threonine). CILP-1, DKK3, SRFP2, or CCN5/WISP2 activity of a variant may be assessed by any method known by the skilled person as described above.

In some preferred embodiments, said CILP-1, DKK3, SRFP2, and CCN5/WISP2 protein or variant comprises or consists of a sequence selected from the group consisting of the sequences SEQ ID NO: 2, 4, 6 and 8 and the sequences having at least 70%, 75%, 80%, 85%, 90%, 95%, 96%, 97%, 98% or 99% identity with any one of said sequences.

In a particular embodiment said transgene may be an optimized sequence, in particular a codon-optimized sequence, encoding CILP-1, DKK3, SRFP2, or CCN5/WISP2 protein or variant thereof.

The term “codon optimized” means that a codon that expresses a bias for human (i.e. is common in human genes but uncommon in other mammalian genes or non-mammalian genes) is changed to a synonymous codon (a codon that codes for the same amino acid) that does not express a bias for human. Thus, the change in codon does not result in any amino acid change in the encoded protein.

According to the invention several modulators of the Wnt and/or TGF-β pathway may be used simultaneously, separately or sequentially for the treatment of cardiomyopaties.

### Nucleic Acid Construct

In a preferred embodiment, said modulator is included in a nucleic acid construct comprising a nucleotide sequence encoding the modulator.

The term “nucleic acid construct” as used herein refers to a man-made nucleic acid molecule resulting from the use of recombinant DNA technology. A nucleic acid construct is a nucleic acid molecule, either single- or double-stranded, which has been modified to contain segments of nucleic acids sequences, which are combined and juxtaposed in a manner, which would not otherwise exist in nature. A nucleic acid construct usually is a “vector”, i.e. a nucleic acid molecule which is used to deliver exogenously created DNA into a host cell.

The nucleic acid construct may comprise or consist of DNA, RNA or a synthetic or semi-synthetic nucleic acid which is expressible in the individual's target cells or tissue (e.g. cells constitutive of the heart or cardiac cells).

Preferably, the nucleic acid construct comprises said sequence encoding the modulator operably linked to one or more control sequences that direct the expression of a transgene in cells constitutive of the heart. Such sequences which are well-known in the art include in particular a promoter, and further regulatory sequences capable of further controlling the expression of a transgene, such as without limitation, enhancer, terminator, intron, silencer, in particular tissue-specific silencer, and microRNA.

The control sequence may include a promoter that is recognized by cardiac cells. The promoter contains transcriptional control sequences that mediate the expression of the modulator upon introduction into a host cell. The promoter may be any polynucleotide that shows transcriptional activity in cells including mutant, truncated, and hybrid promoters. The promoter may be a constitutive or inducible promoter, preferably a constitutive promoter, and more preferably a strong constitutive promoter.

The promoter may also be tissue-specific, in particular specific of cardiac cells. In a particular embodiment, the nucleic acid construct of the disclosure further comprises a cardiac-specific promoter operably-linked to the transgene as described above. In the context of this disclosure, a “cardiac-specific promoter” is a promoter which is more active in the cardiac than in any other tissue of the body. Typically, the activity of a cardiac specific promoter will be considerably greater in the cardiac than in other tissues. For example, such a promoter may be at least 2, at least 3, at least 4, at least 5 or at least 10 times more active (for example as determined by its ability to drive the expression in a given tissue in comparison to its ability to drive the expression in other cells or tissues). Accordingly, a cardiac specific promoter allows an active expression in the cardiac of the gene linked to it and prevents its expression in other cells or tissues.

Examples of suitable promoters include, but are not limited to, human troponin T gene promoter (TNNT2), alpha myosin heavy chain promoter (a-MHC), myosin light chain 2 promoter (MLC-2), alpha-cardiac actin promoter, alpha-tropomyosin promoter, cardiac troponin C promoter, cardiac myosin-binding protein C promoter, sarco/endoplasmic reticulum Ca²⁺ ATPase (SERCA) promoter, desmin promoter, MH promoter, CK8 promoter and MHCK7 promoter. Preferably, the promoter is human cardiac troponin T promoter. The Muscle Hybrid promoter (MH promoter) is disclosed for example in Piekarowicz et al., Molecular Therapy, 2019, 15, 157-169). CK8 is muscle creatine kinase proroter/enhancer element (Goncalves et al., Mol. Ther., 2011, 19, 1331-1341). MHCK7 promoter is based on enhancer/promoter regions of muscle creatine kinase (CK) and alpha-myosin heavy-chain genes (Salva et al., Mol. Ther., 2007, 15, 320-329).

The control sequence may also include appropriate transcription initiation, termination, and enhancer sequences; efficient RNA processing signals such as splicing and polyadenylation signals; sequences that stabilize cytoplasmic mRNA; sequences that enhance translation efficiency (i.e., Kozak consensus sequence); and/or sequences that enhance protein stability. A great number of expression control sequences, e.g., native, constitutive, inducible and/or tissue-specific, are known in the art and may be utilized to drive expression of the nucleic acid sequence encoding CILP-1, DKK3, SRFP2, or CCN5/WISP2. Typically, the transgene encoding CILP-1, DKK3, SRFP2, or CCN5/WISP2 TGF-β is operably linked to a transcriptional promoter and a transcription terminator.

In particular embodiments, the nucleic acid construct comprises an intron, in particular an intron placed between the promoter and the coding sequence. An intron is introduced to increase mRNA stability and protein production. In addition, a modified intron designed to decrease the number of, or even totally remove, alternative open reading frames (ARFs) found in said intron can significantly improve the expression of the transgene.

Apart from the specific delivery systems embodied below in the examples, various delivery systems are known and can be used to administer the nucleic acid construct as described above, e.g., encapsulation in liposomes, microparticles, microcapsules, recombinant cells capable of expressing the CILP-1, DKK3, SRFP2, or CCN5/WISP2coding sequence, receptor-mediated endocytosis, construction of a therapeutic nucleic acid as part of a retroviral or other vector, etc.

In a preferred embodiment, said nucleic acid construct comprises interfering nucleic acid able to repress CILP-1 gene expression comprising at least one sequence selected from sequences SEQ ID NO: 1 to 4. More preferably, said nucleic acid construct comprises four interfering nucleic acid of sequences SEQ ID NO: 1 to 4.

In another preferred embodiment, said nucleic acid construct comprises a transgene encoding CILP-1, DKK3, SRFP2, or CCN5/WISP2 or a variant thereof, according to the present disclosure.

### Expression Vector

The nucleic acid construct as described above may be contained in an expression vector.

The invention may use any vector suitable for the delivery and expression of nucleic acid into individual's cells, in particular suitable for gene therapy, more particularly targeted gene therapy directed to a target tissue or cells in the individual. Such vectors that are well-known in the art include viral and non-viral vectors, wherein said vectors may be integrative or non-integrative; replicative or non-replicative. In some particular embodiments, gene therapy is directed to cardiac cells or tissue.

Non-viral vector includes the various (non-viral) agents which are commonly used to either introduce or maintain nucleic acid into individual's cells. Agents which are used to introduce nucleic acid into individual's cells by various means include in particular polymer-based, particle-based, lipid-based, peptide-based delivery vehicles or combinations thereof, such as with no limitations cationic polymer, dendrimer, micelle, liposome, exosome, microparticle and nanoparticle including lipid nanoparticle (LNP); and cell penetrating peptides (CPP). CPP are in particular cationic peptides such as poly-L-Lysine (PLL), oligo-arginine, Tat peptides, Penetratin or Transportan peptides and derivatives thereof such as for example Pip. Agents which are used to maintain nucleic acid into individual's cells (either integrated into chromosome(s) or else in extrachromosomal form) include in particular naked nucleic acid vectors such as plasmids, transposons and mini-circles, and gene-editing and RNA-editing systems. Transposon includes in particular the hyperactive Sleeping Beauty (SB100X) transposon system (Mates et al. 2009). Gene-editing and RNA-editing systems may use any site-specific endonuclease such as Cas nuclease, TALEN, meganuclease, zinc finger nuclease and the like. In addition, these approaches can advantageously be combined to introduce and maintain the nucleic acid of the invention into individual's cells.

Viral vectors are by nature capable of penetrating into cells and delivering nucleic acid(s) of interest into cells, according to a process named as viral transduction.

As used herein, the term “viral vector” refers to a non-replicating, non-pathogenic virus engineered for the delivery of genetic material into cells. In viral vectors, viral genes essential for replication and virulence are replaced with an expression cassette for the transgene of interest. Thus, the viral vector genome comprises the transgene expression cassette flanked by the viral sequences required for viral vector production.

As used herein, the term “recombinant virus” refers to a virus, in particular a viral vector, produced by standard recombinant DNA technology techniques that are known in the art.

As used herein, the term “virus particle” or “viral particle” is intended to mean the extracellular form of a non-pathogenic virus, in particular a viral vector, composed of genetic material made from either DNA or RNA surrounded by a protein coat, called the capsid, and in some cases an envelope derived from portions of host cell membranes and including viral glycoproteins.

As used herein, a viral vector refers to a viral vector particle.

A preferred vector for delivering the nucleic acid (nucleic acid construct) of the invention is a viral vector, in particular suitable for gene therapy, more particularly gene therapy directed to a target tissue or cells in the individual such as cardiac cells or tissue. In particular, the viral vector may be derived from a non-pathogenic parvovirus such as adeno-associated virus (AAV), a retrovirus such as a gammaretrovirus, spumavirus and lentivirus, an adenovirus, a poxvirus and an herpes virus. The viral vector is preferably an integrating vector such as AAV or lentivirus vector, preferably AAV vector. Lentivirus vector may be pseudotyped with an envelope glycoprotein from another virus for targeting the cells/tissues of interest.

The vector comprises the viral sequences required for viral vector production such as the lentiviral LTR sequences or the AAV ITR sequences flanking the expression cassette.

In particular embodiments, the vector is a particle or vesicle, in particular lipid-based micro- or nano-vesicle or particle such as liposome or lipid nanoparticle (LNP). In more particular embodiments, the nucleic acid is RNA and the vector is a particle or vesicle as described above.

In another particular embodiment, the vector is an AAV vector. AAV vectors have gained considerable interest as vectors for human gene therapy. Among the favourable properties of the virus are its lack of association with any human disease, its ability to infect both dividing and non-dividing cells, and the wide range of cell lines derived from different tissues that can be infected.

The AAV genome is composed of a linear, single-stranded DNA molecule which contains 4681 bases (Berns and Bohenzky, 1987, Advances in Virus Research (Academic Press, Inc.) 32:243-307). The genome includes inverted terminal repeats (ITRs) at each end, which function in cis as origins of DNA replication and as packaging signals for the virus. The ITRs are approximately 145 bp in length. The internal non-repeated portion of the genome includes two large open reading frames, known as the AAV rep and cap genes, respectively. These genes code for the viral proteins involved in replication and packaging of the virion. In particular, at least four viral proteins are synthesized from the AAV rep gene, Rep 78, Rep 68, Rep 52 and Rep 40, named according to their apparent molecular weight. The AAV cap gene encodes at least three proteins, VP1, VP2 and VP3. For a detailed description of the AAV genome, see, e.g., Muzyczka, N. 1992 Current Topics in Microbiol. and Immunol. 158:97-129.

Thus, in one embodiment, the nucleic acid construct or expression vector comprising transgene as described above further comprises a 5′ITR and a 3′ITR sequences, preferably a 5′ITR and a 3′ ITR sequences of an adeno-associated virus.

As used herein the term “inverted terminal repeat (ITR)” refers to a nucleotide sequence located at the 5′-end (5′ITR) and a nucleotide sequence located at the 3′-end (3′ITR) of a virus, that contain palindromic sequences and that can fold over to form T-shaped hairpin structures that function as primers during initiation of DNA replication. They are also needed for viral genome integration into the host genome; for the rescue from the host genome; and for the encapsidation of viral nucleic acid into mature virions. The ITRs are required in cis for the vector genome replication and its packaging into the viral particles.

AAV ITRs for use in the viral vector of the disclosure may have a wild-type nucleotide sequence or may be altered by the insertion, deletion or substitution. The serotype of the inverted terminal repeats (ITRs) of the AAV may be selected from any known human or nonhuman AAV serotype. In specific embodiments, the nucleic acid construct or viral expression vector may be carried out by using ITRs of any AAV serotype, including AAV1, AAV2, AAV3 (including types 3A and 3B), AAV4, AAV5, AAV6, AAV7, AAV8, AAV9, AAV10, AAV11, AAV12, avian AAV, bovine AAV, canine AAV, equine AAV, ovine AAV, and any other AAV serotype or engineered AAV now known or later discovered.

In one embodiment, the nucleic acid construct further comprises a 5′ITR and a 3′ITR of the corresponding capsid, or preferably 5′ITR and a 3′ITR of a serotype AAV-2.

On the other hand, the nucleic acid construct or expression vector of the disclosure can be carried out by using synthetic 5′ITR and/or 3′ITR; and also by using a 5′ITR and a 3′ITR which come from viruses of different serotypes. All other viral genes required for viral vector replication can be provided in trans within the virus-producing cells (packaging cells) as described below. Therefore, their inclusion in the viral vector is optional.

In one embodiment, the nucleic acid construct or viral vector of the disclosure comprises a 5′ITR, a W packaging signal, and a 3′ITR of a virus. “Y packaging signal” is a cis-acting nucleotide sequence of the virus genome, which in some viruses (e.g. adenoviruses, lentiviruses . . . ) is essential for the process of packaging the virus genome into the viral capsid during replication.

The construction of recombinant AAV viral particles is generally known in the art and has been described for instance in U.S. Pat. Nos. 5,173,414 and 5,139,941; WO 92/01070, WO 93/03769, Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; and Kotin, R. M. (1994) Human Gene Therapy 5:793-801.

### Viral Particle

In a preferred embodiment, the present disclosure relates to viral particles packaging a nucleic acid construct or expression vector as described above.

The nucleic acid construct or the expression vector of the disclosure may be packaged into a virus capsid to generate a “viral particle”, also named “viral vector particle”. In a particular embodiment, the nucleic acid construct or the expression vector as described above is packaged into an AAV-derived capsid to generate an “adeno-associated viral particle” or “AAV particle”. The present disclosure relates to a viral particle comprising a nucleic acid construct or an expression vector of the disclosure and preferably comprising capsid proteins of adeno-associated virus.

The term AAV vector particle encompasses any recombinant AAV vector particle or mutant AAV vector particle, genetically engineered. A recombinant AAV particle may be prepared by encapsidating the nucleic acid construct or viral expression vector including ITR(s) derived from a particular AAV serotype on a viral particle formed by natural or mutant Cap proteins corresponding to an AAV of the same or different serotype.

Proteins of the viral capsid of an adeno-associated virus include the capsid proteins VP1, VP2, and VP3. Differences among the capsid protein sequences of the various AAV serotypes result in the use of different cell surface receptors for cell entry. In combination with alternative intracellular processing pathways, this gives rise to distinct tissue tropisms for each AAV serotype.

Several techniques have been developed to modify and improve the structural and functional properties of naturally occurring AAV viral particles (Bunning H et al. J Gene Med, 2008; 10: 717-733; Paulk et al. Mol ther. 2018; 26(1):289-303; Wang L et al. Mol Ther. 2015; 23(12):1877-87; Vercauteren et al. Mol Ther. 2016; 24(6):1042-1049; Zinn E et al., Cell Rep. 2015; 12(6):1056-68).

Thus, in AAV viral particle according to the present disclosure, the nucleic acid construct or viral expression vector including ITR(s) of a given AAV serotype can be packaged, for example, into: a) a viral particle made of capsid proteins derived from the same or different AAV serotype; b) a mosaic viral particle made of a mixture of capsid proteins from different AAV serotypes or mutants; c) a chimeric viral particle made of capsid proteins that have been truncated by domain swapping between different AAV serotypes or variants.

The skilled person will appreciate that the AAV viral particle for use according to the present disclosure may comprise capsid proteins from any AAV serotype including AAV1, AAV2, AAV3 (including types 3A and 3B), AAV4, AAV5, AAV6, AAV7, AAV8, AAV9, AAV10, AAV11, AAV12, AAV2i8, AAVrh10, AAVrh39, AAVrh43, AAVrh74, AAV-LK03, AAV2G9, AAV.PHP, AAV-Anc80, AAV3B and AAV9.rh74 (as disclosed in WO2019/193119).

For gene transfer into human cardiac cells, AAV serotypes 1, 6, 8, 9 and AAV9.rh74 are preferred. The AAV serotype 9 and AAV9.rh74 are particularly well suited for the induction of expression in cells of the myocardium/cardiomyocytes. In a specific embodiment, the AAV viral particle comprises a nucleic acid construct or expression vector of the disclosure and preferably capsid proteins from AAV9 or AAV9.rh74 serotype.

### Pharmaceutical Compositions and Treatment

The Wnt or TGF-β pathway modulator, nucleic acid construct, expression vector or viral particle is preferably used in the form of a pharmaceutical composition comprising a therapeutically effective amount of the Wnt or TGF-β pathway modulator, nucleic acid construct, expression vector or viral particle.

The nucleic acid construct, expression vector or viral particle and derived pharmaceutical composition of the invention may be used for treating diseases by gene therapy, in particular targeted gene therapy directed to cardiac cells or tissue. The pharmaceutical composition of the invention may also be used for treating diseases by cell therapy, in particular cell therapy directed to cardiac cells or tissue.

As used herein “Gene therapy” refers to a treatment of an individual which involves delivery of nucleic acid of interest into an individual's cells for the purpose of treating a disease. Delivery of the nucleic acid is generally achieved using a delivery vehicle, also known as a vector. Viral and non-viral vectors may be employed to deliver a gene to a patient's cells.

As used herein “Cell therapy” refers to a process wherein cells modified by a nucleic or vector of the invention are delivered to the individual in need thereof by any appropriate mean such as for example by intravenous injection (infusion), or injection in the tissue of interest (implantation or transplantation). In particular embodiments, cell therapy comprises collecting cells from the individual, modifying the individual's cells with the nucleic acid or vector of the invention, and administering the modified cells back to the patient. As used herein “cell” refers to isolated cell, natural or artificial cellular aggregate, bioartificial cellular scaffold and bioartificial organ or tissue.

In the context of the invention, a therapeutically effective amount refers to a dose sufficient for reversing, alleviating or inhibiting the progress of the disorder or condition to which such term applies, or reversing, alleviating or inhibiting the progress of one or more symptoms of the disorder or condition to which such term applies. The term “effective dose” or “effective dosage” is defined as an amount sufficient to achieve, or at least partially achieve, the desired effect.

The effective dose is determined and adjusted depending on factors such as the composition used, the route of administration, the physical characteristics of the individual under consideration such as sex, age and weight, concurrent medication, and other factors, that those skilled in the medical arts will recognize.

In the various embodiments of the present invention, the pharmaceutical composition comprises a pharmaceutically acceptable carrier and/or vehicle.

A “pharmaceutically acceptable carrier” refers to a vehicle that does not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

Preferably, the pharmaceutical composition contains vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or suspensions. The solution or suspension may comprise additives which are compatible with viral vectors and do not prevent viral vector particle entry into target cells. In all cases, the form must be sterile and must be fluid to the extent that easy syringe ability exists. It must be stable under the conditions of manufacture and storage and must be preserved against the contaminating action of microorganisms, such as bacteria and fungi. An example of an appropriate solution is a buffer, such as phosphate buffered saline (PBS) or Ringer lactate.

The Wnt or TGF-β pathway modulator, nucleic acid construct, expression vector or viral particle, pharmaceutical composition according to the present invention are used in the treatment of any genetic dilated cardiomyopathy (DCM or CMD).

The treatment of the genetic dilated cardiomyopathies is preferably by gene therapy using a nucleic acid construct, expression vector or viral particle or derived pharmaceutical composition according to the present disclosure.

In genetically-induced dilated cardiomyopathies, most of the genes involved code for structural elements of cardiomyocytes, including extracellular matrix or Golgi apparatus proteins (laminin, fukutin) involved in cellular adhesion and signaling pathways; desmosome proteins (desmocollin, plakoglobin) involved in cellular junctions; sarcoplasmic reticulum proteins (RyR2, SERCA2a, phospholamban) involved in calcium homeostasis; nuclear envelop proteins (lamin A/C) involved in myocardial structural organisation; cytoskeleton proteins (dystrophin, telethonin, a-actinin, desmin, sarcoglycans) involved in cytoskeleton integrity and muscular strength transmission; and sarcomer proteins (titin, troponin, myosin, actin) involved in generation and transmission of muscular strength.

Mutations in many genes have been found to cause different forms of dilated cardiomyopathy (CMD). These include in particular:
CMD1A, dilated cardiomyopathy-1A (OMIM #115200) caused by heterozygous mutation in the lamin A/C gene (LMNA), (OMIM #150330) on chromosome 1q22; or heterozygous mutation in the laminin alpha 2 (LAMA2 or MEROSIN) gene (OMIM #156225; Marques et al., Neuromuscul. Disord., 2014, doi.org/10.10106/);
CMD1B (OMIM #600884) on 9q13; the gene referred as FDC locus was placed in the interval between D9S153 and D9S152. Friedreich ataxia (OMIM #229300), which is frequently associated with dilated cardiomyopathy, maps to the same region as does also the cAMP-dependent protein kinase (OMIM #176893), which regulates calcium-channel ion conductance in the heart. Tropomodulin (OMIM #190930), which maps to 9q22, was a particularly attractive candidate gene.
CMD1C (OMIM #601493) with or without left ventricular noncompaction, caused by mutation in the lim domain-binding 3, LDB3 (or ZASP) gene (OMIM #605906) on 10q23;
CMD1D (OMIM #601494), caused by mutation in the troponin T2, cardiac (TNNT2) gene (OMIM #191045) on 1q32;
CMD1E (OMIM #601154), caused by mutation in the SCN5A gene (OMIM #600163) on 3p22;
CMD1F: The symbol CMD1F was formerly used for a disorder later found to be the same as desmin-related myopathy or myopathy, myofibrillar (MFM) (OMIM #601419);
CMD1G (OMIM #604145), caused by mutation in the titin (TTN) gene (OMIM #188840) on 2q31;
CMD11H (OMIM #604288) on 2q14-q22;
CMD1I (OMIM #604765), caused by mutation in the desmin (DES) gene (OMIM #125660) on 2q35;
CMD1J (OMIM #605362), caused by mutation in the EYA4 gene (OMIM #603550) on 6q23;
CMD1K (OMIM #605582) on 6912-q16;
CMD1L (OMIM #606685), caused by mutation in the sarcoglycan delta (SGCD) gene (OMIM #601411) on 5q33;
CMD1M (OMIM #607482), caused by mutation in the CSRP3 gene (OMIM #600824) on 11p15;
CMD1N; (OMIM #607487) caused by mutation in the TITIN-CAP (telethonin or TCAP) gene (OMIM #604488).
CMD10 (OMIM #608569), caused by mutation in the ABCC9 gene (OMIM #601439) on 12p12;
CMD1P (OMIM #609909), caused by mutation in the phospholamban (PLN) gene (OMIM #172405) on 6q22;
CMD1Q (OMIM #609915) on 7q22.3-q31.1;
CMD1R (OMIM #613424), caused by mutation in the actin alpha, cardiac muscle (ACTC1) gene (OMIM #102540) on 15q14;
CMD1S (OMIM #613426), caused by mutation in the myosin heavy chain 7, cardiac muscle, beta (MYH7) gene (OMIM #160760) on 14q12;
CMD1U (OMIM #613694), caused by mutation in the PSEN1 gene (OMIM #104311) on 14q24;
CMD1V (OMIM #613697), caused by mutation in the PSEN2 gene (OMIM #600759) on 1q42;
CMD1W (OMIM #611407), caused by mutation in the gene encoding metavinculin (VCL; OMIM #193065) on 10q22;
CMD1X (OMIM #611615), caused by mutation in the gene encoding fukutin (FKTN; OMIM #607440) on 9q31;
CMD1Y (OMIM #611878), caused by mutation in the TPM1 gene (OMIM #191010) on 15q22;
CMD1Z (OMIM #611879), caused by mutation in the troponin C (TNNC1) gene (OMIM #191040) on 3p21;
CMD1AA (OMIM #612158), caused by mutation in the actinin alpha-2 (ACTN2) gene (OMIM #102573) on 1q43;
CMD1BB (OMIM #612877), caused by mutation in the DSG2 gene (OMIM #125671) on 18q12;
CMD1CC (OMIM #613122), caused by mutation in the NEXN gene (OMIM #613121) on 1p31;
CMD1DD (OMIM #613172), caused by mutation in the RNA binding motif protein 20 (RBM20) gene (OMIM #613171) on 10q25;
CMD1EE (OMIM #613252), caused by mutation in the myosin heavy chain 6, cardiac muscle, alpha (MYH6) gene (OMIM #160710) on 14q12;
CMD1FF (OMIM #613286), caused by mutation in the troponin I, cardiac (TNNI3) gene (OMIM #191044) on 19q13;
CMD1GG (OMIM #613642), caused by mutation in the SDHA gene (OMIM #600857) on 5p15;
CMD1HH (OMIM #613881), caused by mutation in the BCL2-associated athanogene 3 (BAG3) gene (OMIM #603883) on 10q26;
CMD1II (OMIM #615184), caused by mutation in the CRYAB gene (OMIM #123590) on 6921;
CMD1JJ (OMIM #615235), caused by mutation in the laminin alpha 4 (LAMA4) gene (OMIM #600133) on 6921;
CMD1KK (OMIM #615248), caused by mutation in the MYPN gene (OMIM #608517) on 10q21;
CMD1LL (OMIM #615373), caused by mutation in the PRDM16 gene (OMIM #605557) on 1p36;
CMD1MM (OMIM #615396), caused by mutation in the MYBPC3 gene (OMIM #600958) on 11p11;
CMD1NN (OMIM #615916), caused by mutation in the RAF1 gene (OMIM #164760) on 3p25;
CMD2A (OMIM #611880), caused by mutation in the troponin I, cardiac (TNNI3) gene on 19q13;
CMD2B (OMIM #614672), caused by mutation in the GATAD1 gene (OMIM #614518) on 7921;
CMD2C (OMIM #618189), caused by mutation in the PPCS gene (OMIM #609853) on 1p34;
CMD3A, a previously designated X-linked form was found to be the same as Barth syndrome (OMIM #302060); and
CMD3B (OMIM #302045), an X-linked form of CMD, caused by mutation in the dystrophin gene (DMD, OMIM #300377).

Desmin-related myopathy or myopathy, myofibrillar (MFM) (OMIM #601419). is a noncommittal term that refers to a group of morphologically homogeneous, but genetically heterogeneous chronic neuromuscular disorders. The morphologic changes in skeletal muscle in MFM result from disintegration of the sarcomeric Z disc and the myofibrils, followed by abnormal ectopic accumulation of multiple proteins involved in the structure of the Z disc, including desmin, alpha-B-crystallin (CRYAB; OMIM #123590), dystrophin (OMIM #300377), and myotilin (TTID; OMIM #604103). Myofibrillar myopathy-1 (MFM1) is caused by heterozygous, homozygous, or compound heterozygous mutation in the desmin gene (DES; OMIM #125660) on chromosome 2q35. Other forms of MFM include MFM2 (OMIM #608810), caused by mutation in the CRYAB gene (OMIM #123590); MFM3 (OMIM #609200) (OMIM #182920), caused by mutation in the MYOT gene (OMIM #604103); MFM4 (OMIM #609452), caused by mutation in the ZASP gene (LDB3; OMIM #605906); MFM5 (OMIM #609524), caused by mutation in the FLNC gene (OMIM #102565); MFM6 (OMIM #612954), caused by mutation in the BAG3 gene (OMIM #603883); MFM7 (OMIM #617114), caused by mutation in the KY gene (OMIM #605739); MFM8 (OMIM #617258), caused by mutation in the PYROXD1 gene OMIM #617220); and MFM9 (OMIM #603689), caused by mutation in the TTN gene (titin; OMIM #188840).

Mutations in other genes have also been found to cause different forms of dilated cardiomyopathy. These include:
desmocollin 2 (DSC2, OMIM #125645) responsible for Arrhythmogenic Right Ventricular Dysplasia 11 (OMIM #610476) and dilated cardiomyopathy (Elliott et al., Circ. Vase. Genet., 2010, 3, 314-322);
junctiun plakoglobin (JUP or plakoglobin; OMIM #173325) responsible for Arrhythmogenic Right Ventricular Dysplasia 12 (OMIM #611528) and dilated cardiomyopathy (Elliott et al., Circ. Vase. Genet., 2010, 3, 314-322);
ryanodine receptor 2 (RYR2; OMIM #180902) responsible for Arrhythmogenic Right Ventricular Dysplasia 2 (OMIM #600996) and Ventricular tachycardia, catecholaminergic polymorphic 1 (OMIM #604772) and dilated cardiomyopathy (Zahurul, Circulation, 2007, 116, 1569-1576);
ATPase, Ca(2+)-transporting slow-twitch (ATP2A2; ATP2B, sarcoplasmic reticulum Ca(2+) ATPase 2 isoform alpha (SERCA2a); and
emerin (EMD); fukutin-related protein (FKRP); tafazzin (TAZ); desmoplakin (DSP); and Sodium Channels such as SCN1B, SCN2B, SCN3B, SCN4B, SCN4A, SCN5A and others.

In some embodiments, the genetic dilated cardiomyopathy is caused by mutation(s) in a gene selected from the group consisting of: laminin, in particular laminin alpha 2 (LAMA2) and laminin alpha 4 (LAMA4); emerin (EMD); fukutin (FKTN); fukutin-related protein (FKRP); desmocollin, in particular desmocollin 2 (DSC2); plakoglobin (JUP); ryanodine receptor 2 (RYR2); sarcoplasmic reticulum Ca(2+) ATPase 2 isoform alpha (SERCA2a); phospholamban (PLN); lamin A/C (LMNA); dystrophin (DMD); TITIN-CAP or telethonin (TCAP); actinin, in particular actinin alpha-2 (ACTN2); desmin (DES); actin, in particular cardiac actin, actin alpha, cardiac muscle (ACTC1); sarcoglycan, in particular sarcoglycan delta (SGCD); titin (TTN); troponin, in particular cardiac troponin, troponin T2, cardiac (TNNT2); troponin C (TNNC1) and troponin I, cardiac (TNNI3); myosin, in particular myosin heavy chain 7, cardiac muscle, beta (MYH7) and myosin heavy chain 6, cardiac muscle, alpha (MYH6); RNA binding motif protein 20 (RBM20); BCL2-associated athanogene 3 (BAG3); desmoplakin (DSP); tafazzin (TAZ) and sodium channels such as SCN1B, SCN2B, SCN3B, SCN4B, SCN4A, SCN5A and others.

In some particular embodiments, the genetic dilated cardiomyopathy is caused by mutation(s) in a gene selected from the group consisting of: laminin, in particular laminin alpha 2 (LAMA2) and laminin alpha 4 (LAMA4); emerin (EMD); fukutin (FKTN); fukutin-related protein (FKRP); desmocollin, in particular desmocollin 2 (DSC2); plakoglobin (JUP); ryanodine receptor 2 (RYR2); sarcoplasmic reticulum Ca(2+) ATPase 2 isoform alpha (SERCA2a); phospholamban (PLN); dystrophin (DMD); TITIN-CAP or telethonin (TCAP); actinin, in particular actinin alpha-2 (ACTN2); desmin (DES); actin, in particular cardiac actin, actin alpha, cardiac muscle (ACTC1); sarcoglycan, in particular sarcoglycan delta (SGCD); titin (TTN); troponin, in particular cardiac troponin, troponin T2, cardiac (TNNT2); troponin C (TNNC1) and troponin I, cardiac (TNNI3); myosin, in particular myosin heavy chain 7, cardiac muscle, beta (MYH7) and myosin heavy chain 6, cardiac muscle, alpha (MYH6); RNA binding motif protein 20 (RBM20); BCL2-associated athanogene 3 (BAG3); desmoplakin (DSP); tafazzin (TAZ) and sodium channels such as SCN1B, SCN2B, SCN3B, SCN4B, SCN4A, SCN5A and others. Preferably, dystrophin (DMD) or titin (TTN).

The invention provides also a method for treating a genetic dilated cardiomyopathy according to the present disclosure, comprising: administering to a patient a therapeutically effective amount of the Wnt or TGF-β modulator, nucleic acid construct, expression vector, viral particle, or combination thereof, or the pharmaceutical composition according to the present disclosure.

A further aspect of the invention relates to the Wnt or TGF-β pathway modulator, nucleic acid construct, expression vector, viral particle or combination thereof, or the pharmaceutical composition according to the present disclosure, for use in the manufacture of a medicament for the treatment of genetic dilated cardiomyopathies.

A further aspect of the invention relates to the use of the Wnt or TGF-β pathway modulator, nucleic acid construct, expression vector, viral particle or combination thereof, or the pharmaceutical composition according to the present disclosure, for the treatment of genetic dilated cardiomyopathies.

A further aspect of the invention relates to a pharmaceutical composition for treatment of genetic dilated cardiomyopathies according to the present disclosure, comprising the Wnt or TGF-β pathway modulator, nucleic acid construct, expression vector, viral particle or combination thereof, as an active compound.

A further aspect of the invention relates to a pharmaceutical composition comprising the Wnt or TGF-β pathway modulator, nucleic acid construct, expression vector, viral particle or combination thereof, according to the present disclosure for treating a genetic dilated cardiomyopathies according to the present disclosure.

As used herein, the term “patient” or “individual” includes human and other mammalian subjects that receive either prophylactic or therapeutic treatment. Preferably, a patient or individual according to the invention is a human.

Treatment”, or “treating” as used herein, is defined as the application or administration of a therapeutic agent or combination of therapeutic agents (e.g., a Wnt pathway inhibitor and/or a TGF-β pathway inhibitor) to a patient, or application or administration of said therapeutic agents to an isolated tissue or cell line from a patient, who has a genetic dilated cardiomyopathy with the purpose to cure, heal, alleviate, relieve, alter, remedy, ameliorate, improve or affect the genetic dilated cardiomyopathy, or any symptom of the genetic dilated cardiomyopathy. In particular, the terms “treat’ or treatment” refers to reducing or alleviating at least one adverse clinical symptom associated with genetic dilated cardiomyopathy, e.g., cardiac dilatation, in particular left ventricle dilatation and reduced systolic function (e.g., reduced ejection fraction).

The term “treatment” or “treating” is also used herein in the context of administering the therapeutic agents prophylactically.

The pharmaceutical composition of the present invention, is generally administered according to known procedures, at dosages and for periods of time effective to induce a therapeutic effect in the patient. The pharmaceutical composition may be administered by any convenient route, such as in a non-limiting manner by infusion or bolus injection, by absorption through epithelial or mucocutaneous linings (e.g., oral mucosa, rectal and intestinal mucosa, etc.). Administration can be systemic, local, or systemic combined with local; systemic includes parenteral and oral, and local includes local and loco-regional. Systemic administration is preferably parenteral such as subcutaneous (SC), intramuscular (IM), intravascular such as intravenous (IV) or intraarterial; intraperitoneal (IP); intradermal (ID), epidural or else. The parenteral administration is advantageously by injection or perfusion.

The Wnt or TGF-β pathway modulator, nucleic acid construct, expression vector or viral particle, or the pharmaceutical composition according to the present disclosure may be used in combination with other therapeutic active agents, wherein the combined use is by simultaneous, separate or sequential administration.

The practice of the present invention will employ, unless otherwise indicated, conventional techniques, which are within the skill of the art. Such techniques are explained fully in the literature.

The invention will now be exemplified with the following examples, which are not limitative, with reference to the attached drawings in which:

### FIGURE LEGENDS

FIG. 1: qPCR analysis of several deregulated genes in the RNAseq (n=4 per group. Student test).

FIG. 2: Expression of transgenes

A) Relative RT-qPCR abundance of the hCILP transgene in DeltaMex5 mice injected or not injected with the AAV9-hTnnt2-hCILP vector. B) Relative RT-qPCR abundance of the GFP transgene in DeltaMex5 mice injected or not injected by the vector AAV9-4in1shRNA-mLTBP2-GFP. Student test and GFP. n=4. Student's test.

FIG. 3: Morphological analysis.

A) Total mass of mice. B) Measurement of cardiac hypertrophy: heart mass/total mouse mass (%)

FIG. 4: Histological characterization of the heart after treatment with AAV9-hTnnt2-hCILP or AAV-shLTBP2 in DeltaMex5 mice and controls injected with PBS. A) HPS staining of the heart. B) Sirius red staining of the heart. Scale, 500 μm. C) Quantification of the proportion of fibrosis types compared to healthy tissue. Student's test.

FIG. 5: Comparison of DCM marker (Left ventricle mass) measured in ultrasound between C57BL/6 mice, DeltaMex5 mice and DeltaMex5 mice injected with the CILP and shLBTP2 vectors. Student test.

FIG. 6: RT-qPCR measurement of different RNA markers of cardiac involvement. Measurements expressed as a ratio to the C57BL/6 mouse. Student's test.

FIG. 7: RT-qPCR measurement of various RNA markers of cardiac fibrosis. Measurements expressed as a ratio to the C57BL/6 mouse. Student's test.

FIG. 8: Expression of WISP2, DKK3 and SFRP2 transgenes.

Relative RT-qPCR abundance of the transgenes hWISP2, hDKK3, hSFRP2 in DeltaMex5 mice injected or not injected by the vectors AAV9-hTnnt2-hWISP2, AAV9-hTnnt2-hDKK3 and AAV9-hTnnt2-hSFRP2 respectively. Measurements expressed as a ratio to C57BL/6 mice. n=4. Student's test.

FIG. 9: Histological characterization of the heart after treatment with AAV9-hTnnt2-hWISP2, DKK3 or SFRP2 in DeltaMex5 mice. Quantification of the proportion of fibrosis types compared to healthy tissue. Student's test.

FIG. 10: RT-qPCR measurement of different RNA markers of cardiac and fibrotic involvement in the WNT study.

Measurements expressed as a ratio to the C57BL/6 mouse. Student's test.

### EXAMPLES

### 1. Material and Methods

### 1.1 Mouse Models

The mice used in this study were male titin^{Mex5-/Mex5-} (DeltaMex5) and DBA/2J-mdx (DBA2mdx) strains, and their respective controls, strains C57BL/6 and DBA/2. DeltaMex5 mice have the deletion of the penultimate exon (Mex5) of the titin gene (titin^{Mex5-/Mex5-}; Charton et al., Human molecular genetics, 2016, 25, 4518-4532). DBA2mdx mice are a model of Duchenne muscular dystrophy due to a point mutation on exon 23 of the dystrophin gene. DBA2mdx mice are on a DBA/J background which has a mutation on the LTBP4 gene, a protein that regulates the activity of the TGF signaling pathway-O (Fukada, et al. 2010. Am J Pathol 176, 2414-2424). All the mice are handled in accordance with the European directives for the care and use of laboratory animals by humans, and the animal experimentation has been approved by the Ethics Committee for Animal Experimentation C2AE-51 of Evry under the numbers of Project Authorisation Application 2015-003-A and 2018-024-B.

### 1.2 Muscle Sampling and Freezing

The muscles of interest are collected, weighed and frozen in liquid nitrogen (samples for molecular biology analysis) or in cooled isopentane (samples for histology), after being placed transversely or longitudinally on a piece of cork coated with gum arabic. The hearts are frozen in diastole before being frozen with a diluted butanedione solution (5 mM) in tyrode. The samples are then stored at −80° C. until use. For the Sirius Red Fibrosis observation protocol on the whole heart, the hearts are included whole in paraffin and stored at room temperature. For the transparency protocols, the sampled hearts are stored whole in 4% para formaldehyde and kept at +4° C.

### 1.3 RNA Extraction and Quantification

Frozen isopentane muscle is cut into 30 μm thick slices on a cryostat (LEICA CM 3050) at −20° C., separated into eppendorf tubes of about 10-15 slices and stored at −80° C. The TRIzol® method for the extraction of total RNA, based on the solvency properties of nucleic acids in organic solvents, is used. The muscle recovery tubes are resupplied with 0.8 mL of TRIzol® (ThermoFisher) supplemented with glycogen (Roche) at a rate of 0.5 μL/mL of TRIzol®. The tubes are placed in the FastPrep-24 (Millipore) homogenizer for a 20s, 4 m.s. cycle. To recover nucleic acids, after a 5-minute incubation on ice, 0.2 mL of chloroform (Prolabo) is added and mixed with TRIzol®. After a 3-minute incubation at room temperature, the two phases, aqueous and organic, are separated by centrifugation at 12000 g for 15 minutes at 4° C. The aqueous phase, containing the nucleic acids, is removed and placed in a new tube. The RNAs are then precipitated by the addition of 0.5 mL isopropanol (Prolabo) followed by a 10-minute incubation at room temperature and centrifugation for 15 minutes at 12000 g at 4° C. The nucleic acid pellet is washed with 0.5 mL 75% ethanol (Prolabo) and again centrifuged for 10 minutes at 12000 g at 4° C. and then air-dried. The nucleic acids are taken up in 50 μL of nuclease free water, 20 μL are set aside for viral DNA analysis, 30 μL are added to RNAs in (Promega) diluted at 1/50 to preserve the RNAs from degradation. The RNAs are then treated with TURBO Dnase (Ambion) to remove residual DNA. A double Dnase treatment is performed for samples intended for sequencing.

For transcriptome analysis specific to signaling pathways, RT2 Profiler PCR Array (Qiagen) plates are used. The screening plates require the use of a compatible RNA extraction kit, the RNeasy Mini Kit (Qiagen) which extracts the RNA on columns, the kit is used following the supplier's instructions, and the RNAs are then processed by Free DNAse RNAse (Qiagen).

An OD reading is then taken on the ND-8000 spectrophotometer (Nanodrop), from 2 μL of RNA to determine their concentration. RNA is stored at −80° C. and DNA at −20° C.

### 1.4 Measurement of RNA Quality

In the case of RNAs prepared for sequencing, the quality of the RNAs is measured on the Bioanalyzer 2100 (Agilent) which performs capillary electrophoresis of nucleic acids and then their analysis. The quality is visualized by the retention rate and the concentration of the sample in the form of electrophoregrams. A quality score expressed in RIN (for RNA Integrity Number) is calculated for each sample, on a scale of 0 to 10. The RNA Nano chip (Agilent) is used according to the supplier's instructions. A size marker (RNA 6000 Nano Ladder, Agilent) is passed first, to allow evaluation of RNA size in the samples. A marker is added to each sample, emerging at a defined size. For each sample, 1 μL of RNA is deposited on the chip. On the RNA electrophoregram, the ribosomal RNA peaks are observed: 28S (around 4000 nt), 18S (around 2000 nt) and 5S (around 100 nt). The internal marker emerges at the 25 nt position. The INR is calculated as a function of the height and position of the 18S and 28S peaks, the ratio between the 5S, 18S and 28S peaks, and the signal-to-noise ratio. For RNA-seq, the required quality requires an INR of at least 7.

### 1.5 Real-Time Quantitative PCR

Genomic and viral DNA are quantified by qPCR and gene expression by Real-time quantitative PCR. Reverse transcription step is performed on the entire messenger RNA using the RevertAid H Minus First Strand cDNA Synthesis Kit (Thermo-Fisher). Two types of oligonucleotides: so-called “random” hexamers, containing random sequences, and “dT” oligonucleotides, deoxy-thymine polymers, which hybridize to the polyA sequences, making it possible to generate cDNAs in their entirety. The mix used is shown in Table 1.

TABLE 1
Reaction mixture for reverse transcription.

| Product | Quantity | |
|---|---|---|
| RNA | 1 | μg |
| Random hexameres + 1/10 | 50 | ng |
| OligodT | | |
| Reaction buffer 5X | 1/5 | |
| dNTP | 500 μM of each | |
| Ribolock Rnase Inhibitor 40 U/μl | 0.25 | U |
| RevertAid H-Minus 200 U/μL | 200 | U |

| | |
|---|---|
| Water | qsp 20 μL |

The mixture is placed in a thermal cycler for the following cycle: 10 min at 25° C., then 1h15 at 42° C., temperature of action of the enzyme, then the enzyme is inactivated 10 min at 70° C. The cDNAs are stored at +4° C. in the short term or at −20° C. in the long term.

Real-time quantitative PCR is performed either on genomic or viral DNA for vector titration and measurement of vector copy number in tissues, or on cDNA obtained from RNA for quantification of transcripts. It is performed on the LightCycler 480® (Roche) 384-well plate. The nuclease activity of the Thermo-Start DNA Polymerase enzyme contained in ABsolute QPCR ROX Mix (ThermoFisher) allows the detection of PCR products at each amplification cycle by release of a fluorescent reporter. This fluorescent reporter is a fluorophore (FAM, for 6-carboxyfluorescein or VIC, for 2′-chloro-7′phenyl-1,4-dichloro-6-carboxy-fluorescein) is located 5′ from the nucleotide probe which is also labelled with a quencher (TAMRA, for tetramethylrhodamine) in 3′. Separation of the reporter and the quencher results in the fluorescence of the reporter, which is measured by the apparatus. The mixtures for each gene of interest are composed of the two oligos F (forward, sense) and R (reverse, antisense) at 0.2 mM and the corresponding 0.1 mM probe. Commercial mixtures of 20× Taqman Gene Expression Assay (ThermoFisher) primers corresponding to the mRNAs to be quantified bearing the FAM reporter are used (Erreur !Source du renvoi introuvable.). The ribophosphoprotein acid gene RPLPO coding for a ribosomal protein, invariant under the different conditions, was chosen as the normalizing gene using the VIC reporter. The primers and Taqman probe used for amplification of RPLPO are as follows: m181PO.F (5′-CTCCAAGCAGATGCAGCAGA-3′; SEQ ID NO: 15), m267PO.R (5′-ACCATGATGATGCG CAAGGCCAT-3′; SEQ ID NO: 16) and m225PO.P (5′-CCGTGGTGCTGATGGGGGGCAAGA A-3′; SEQ ID NO: 17). DNA samples are either cDNA samples obtained after reverse transcription or viral DNA. The PCR reaction takes place in 384-well plates, each well is duplicated in the quantities shown in the Table 2.

TABLE 2
Reaction mixture for quantitative PCR.

| Product | Quantity |
|---|---|
| DNA | 50 ng |
| Thermo Scientific Absolute qPCR ROX | 1X |
| Mix | |
| TaqMan Gene Expression 20X FAM | 0.5X |
| Standardizer RPLP0 20X VIC | 0.5X |
| Water | qsp 10 μL |

The following PCR program is applied: pre-incubation 15 minutes at 95° C., then 45 amplification cycles of 15 seconds at 95° C. followed by 1 minute at 60° C. using the LightCycler480 (Roche).

TABLE 3
List of Taqman Gene Expression primers used.

| Gene | Reference | Gene | Reference |
|---|---|---|---|
| miR 142-3p | hsa-miR-142-3p | Tgfb1 | Mm01178820_m1 |
| miR 21 | hsa-miR-21 | Ctnnb1 | Mm004893039_m1 |
| miR 31 | mmu-miR-31 | mCilp | Mm00557687_m1 |
| Col1a1 | Mm00801666_g1 | hCilp | Hs01548460_m1 |
| Myh8 | Mm01329494_m1 | GFP | Mr 03989638 |
| Tmem8c | Mm00481256_m1 | mLtbp2 | Mm01307379_m1 |
| Nppa | Mm01255747_g1 | hLtbp2 | Hs00166367_m1 |
| Myh7 | Mm0060555_m1 | mWisp2 | Mm00497471_m1 |
| Myh6 | Mm00440359 m1 | hWisp2 | Hs1031984_m1 |
| Fn | Mm01256744_m1 | mDkk3 | Mm00443800_m1 |
| Vim | Mm01333430_m1 | hDkk3 | Hs00247429_m1 |
| Col1a1 | Mm00801666_g1 | mSfrp2 | Mm01213947_m1 |
| Col3a1 | Mm00802300_m1 | hSfrp2 | Hs00293258_m1 |
| Timp1 | Mm01341361_m1 | | |

The cycle quantification is calculated with the LightCycler® 480 SW 1.5.1 software (Roche) using the maximum second derivative method. Quantitative PCR results are expressed in terms of “Cq”, the number of cycles after which a threshold fluorescence value is reached. This value is then normalized to the value obtained for the reference gene RPLPO.

Mitochondrial PCR kits (PAMM-087Z) and WNT (PAMM-243Z) and TGF-B (PAMM-235Z) target screening are used according to the manufacturer's instructions (RT2 Profiler PCR Arrays, Qiagen). RNA extraction is performed from frozen tissue using the RNasy® Micraoarray tissue kit (Qiagen) and processed with the RNase-Free DNase set (Qiagen). The cDNA is obtained from 500 ng RNA using the RT2 first strand kit (Qiagen) and is used as a template for PCR. The qRT-PCR is performed using the LightCycler480 (Roche, Basel, Switzerland).

### 1.6 RNA Sequencing

### 1.6.1 RNAseq

The samples used for sequencing are total RNA extracted with TRIzol, treated twice with DNAse and having an INR quality >7. 7. Samples of 2pg RNA at 100ng/μL were sent for sequencing to Karolinka Institute. The sequencing library used was prepared with the TruSeq Stranded Total RNA Library Prep Kit (Illumina) and sequencing was performed according to the Illumina protocol. The reads are associated using Fastq-pair and aligned to the mouse genome (mm10) using STAR align. The number of reads is proportional to the abundance of corresponding RNAs in the sample. The sequencing platform then provides several files per sample, containing the alignment files in bam format, the list of genes identified with the number of reads for each sample compared and the list of genes accompanied by a normalized numerical count value expressed in fragments per kb per million reads (FPKM).

### 1.6.2 Analysis

Once the files containing the lists of sequenced transcripts were received, the first step in comparing the samples with each other was to merge the files of the different samples. The goal is to obtain a single table containing, for each transcript identified in the study, its number of reads in each sample. Then, an analysis under the R software was performed with the DESeq2 package: from the number of reads, the samples are normalized, and the differential gene expression for each sample is calculated with respect to its control. The expression difference values (or fold change) are expressed in binary logarithm (log 2.FC), they are associated with their adjusted Pvalue padj. Then, a sorting step was performed to remove: genes containing less than 10 reads under all conditions, genes with no significant pad, genes with a log 2.FC between −0.5 and 0.5 for all conditions. The final table was used to identify genes expressed significantly differentially between the different conditions.

### 1.6.3 Graphic Representation

The alignment of the reads on the mouse genome (mm10) can be observed by viewing the bam files with the Integrative Genomic Viewer (IGV) software. Different R packages are used for the graphical representation of RNAseq results. For Venn diagrams, the VennDiagram package is used. For Volcano Plots, the ggplot2 package is used. The Ingenuity Pathway Analysis software (IPA, Qiagen) and the gene ontology classification system PANTHER are used to visualize the deregulated signaling pathways in the dataset.

### 1.7 Histology

Frozen isopentane muscle is cut into 8 μm thick slices on a cryostat (LEICA CM 3050) at −20° C. The slices are placed on a blade and stored at −80° C.

### 1.7.1 Haematoxylin-Phloxine-Safran Staining

The Hematoxylin-Phloxine-Safran (HPS) marking allows the general appearance of the muscle to be observed and the different tissue and cell structures to be highlighted. Haematoxylin colours nucleic acids dark blue, phloxine colours the cytoplasm pink, saffron colours collagen red-orange.

Cross sections are stained with Harris hematoxylin (Sigma) for 5 min. After washing with water for 2 min, the slides are immersed in a 0.2% (v/v) hydrochloric alcohol solution for 10 s to remove excess stain. After being washed again with water for 1 min, the tissues are blued in a Scott water bath (0.5 g/l sodium bicarbonate and 20 g/l magnesium sulphate solution) for 1 min before being rinsed again with water for 1 min and stained with phloxine 1% (w/v) (Sigma) for 30 s. After rinsing with water for 1 min 30 s, the cuts are dehydrated with 700 ethanol for 1 min and then rinsed in absolute ethanol for 30 s. The tissues are then stained with saffron 1% (v/v in absolute ethanol) for 3 min and rinsed in absolute ethanol. Finally, the cuts are thinned in a Xylene bath for 2 min and then mounted with a slide in the Eukitt medium. Image acquisition is performed with objective 10 on a Zeiss AxioScan white light microscope coupled to a computer and a motorized stage.

From HPS coloured sections, the centronucleation index is calculated by the ratio of the number of centronucleated fibres to the area of the section in mm2.

### 1.7.2 Sirius Red Coloration

This staining allows the collagen fibres to be coloured red and to highlight the presence of fibrotic tissue. Cytoplasms are stained yellow.

Cross sections are dehydrated with acetone for 1 hour for frozen cuts or dewaxed with heat and toluene baths. They are then fixed with 4% formaldehyde for 5 min then 10 min in a Bouin solution. After two washes with water, the slides are immersed in Sirius Red solution (0.1 g Sirius Red per 100 mL picric acid solution) for 1 h for staining. After rinsing with water for 1 min 30 s, the slices are dehydrated in successive ethanol baths: 700 ethanol for 1 min, 950 ethanol for 1 min, absolute ethanol for 1 min and then a second absolute ethanol bath for 2 min. Finally, the slices are thinned in two Xylene baths for 1 min and then mounted with a lamella in the Eukitt medium. Image acquisition is carried out with objective 10 on a Zeiss AxioScan white light microscope coupled to a computer and a motorized stage.

The polarized light images were acquired using a modified right LEICA microscope with a polarizer placed before the sample (Polarizer), along the path of the light; and another polarizer placed after the sample (Anlayzer), which can be rotated by hand, giving the possibility to observe both transmitted and polarized light at the same time. The main axes of the polarizers are oriented at 90 degrees to each other. Polarized light maps have been acquired using a Retiga 2000 CCD sensor (QImaging) coupled to the Cartograph software (Microvision, France). In summary, the light passes through the first polarizer before reaching the sample, the collagen being birefringent the light that passes through it is separated into two rays, which once passed through the second polarizer will allow the differential observation of the two types of Sirius Red and the rest of the cardiac tissue.

### 1.7.3 Quantification of Sirius Red

### 1.7.3.1 Sirius Quant

Sirius Quant is an internally developed ImageJ pluggin (Schneider et al., Nature methods, 2012, 9, 671-675). It is a thresholding macro that allows to isolate and quantify the pixels of the image that are colored red. It works in 3 steps: the first one is to convert the image to black and white. The images resulting from the Red Sirius colorations are very contrasted, so a simple black and white conversion is enough to keep all the useful information. The second one is a very rough thresholding in order to keep only the colored pixels of the image, in other words the pixels belonging to the whole cut. Using the Analyze Particles function with an adapted object size allows automatic detection of the outline of the slice, which is then stored. The third step is a manual thresholding by the user which allows to keep only the pixels colored in red, those associated with the marking. A manual correction tool makes it possible either to remove areas that would have been detected and that are not marking (dust, cut fold, etc.), or to add areas that would not have been taken into account. Once the thresholded image is satisfactory, the number of thresholded pixels and the total number of pixels in the entire section are then measured. A ratio between these two numbers finally gives the fibrosis index in the slice.

### 1.7.3.2 Weka

The images were processed using an artificial intelligence algorithm via the WEKA plugin (ImageJ). The WEKA classifier pluggin was implemented using a training data set containing 17 images representative of the different conditions to be classified. The classes were assigned to healthy tissue (yellow), to both types of staining and to slice rupture (white). The original mappings are mosaic images with a size of approximately 225 megapixels (15k×15k), which were divided into 400 frames (20 rows, 20 columns), each frame measuring approximately 750×750 pixels. Each frame is classified independently and the complete image is then reconstructed. The number of pixels in each class is measured. The total number of pixels belonging to the heart is calculated as the sum of the healthy tissue and the two types of dye uptake. The ratio of each class is then calculated by dividing the number of pixels in the class by the total number of pixels in the heart.

### 1.7.3.3 Whole-Heart Reconstruction and Quantification

Sections of a whole heart colored by Sirius Red were scanned with a scanner (Axioscan ZI, Zeiss) with a 10× lens. A total of 483 images were obtained. They were aligned using ImageJ's pluggin: Linear Stack Alignment with SIFT (Lowe et al., International Journal of Computer Vision, 2004, 60, 91-110). Some images were manually aligned when the software did not allow a satisfactory alignment. The image was loaded into Imaris (BitPlane, USA) for reconstruction and 3D visualization. Once the images were aligned, the Sirius Quant pluggin in fully automatic mode using Otsu thresholding (Otsu N, Cybernetics, 1979, 9, 62-66) resulted in 483 fibrosis ratio values corresponding to each image. These values were filtered using the sliding average method, which is a method of reducing noise in a signal to avoid the errors inherent in automating an algorithm. The use of the moving average allows to limit these errors by replacing each fibrosis ratio of an image by the average of itself, the ratio of the image preceding it and the ratio of the image following it.

### 1.7.4 Fluorescence Immuno-Histo Labeling

The slides are taken out of the freezer and allowed to dry at room temperature for 10 minutes, after which the cuts are wrapped with DAKOpen. The slices are then rehydrated for 5 min in PBS 1×. If the protein of interest is located in the nucleus, the slices are permeabilised for 15 min in a 0.3% triton solution in PBS 1×, then washed 3 times in PBS for 5 min. The slices are then saturated with 10% goat serum, 10% fetal calf serum, PBS 1× for 30 min at room temperature in a humidity chamber. The saturation medium is replaced by the primary antibody solution diluted in PBS 1×+10% blocking solution overnight at 4° C. in a wet chamber. Four successive washes in 1×PBS for 5 minutes are performed before hybridizing with the secondary antibody solution coupled with an Alexa 488 or 594 (1/1000) fluorochrome coupled fluorochrome in 1×PBS+10% blocking solution for 1 h at room temperature in a light-protected wet chamber. A final series of four 5-minute washes in PBS 1× is performed and a fluoromount slide assembly containing DAPI is performed. The sections are then visualized using a fluorescence microscope (Zeiss AxioScan or Leica TCS-SP8 confocal microscope).

TABLE 4
List of antibodies used in immunohistology.

| Antibody | Species | Supplier | Reference | Dilution |
|---|---|---|---|---|
| Collagen I | Mouse | Abcam | ab6308 | 1/100 |
| Collagen III | Rabbit | Abcam | ab7778 | 1/100 |
| Fibronectin | Mouse | Sigma | F7387 | 1/200 |
| Vimentine | Mouse | Chemicon | MAB3400 | 1/100 |
| Vinculine | Mouse | Sigma | V9131 | 1/100 |
| Actin F | Mouse | Abcam | ab205 | 1/100 |
| Titine N2B | Rabbit | Myomedix | #6678 | 1/75 |
| Titanium M8M9 | Rabbit | Myomedix | #3375 | 1/75 |
| Titanium IS7-1 | Rabbit | Genescript | LVEEPPPREVVLKTSC | 1/2 |
| M10-1 Titanium | Rabbit | Genescript | IEALPSDISIDEGKV | 1/75 |
| α-synemin | Rabbit | SantaCruz | sc-68849 | 1/25 |
| Obscurine | Rabbit | Atlas Antibody | HPA040066 | 1/50 |
| Myosprin | Rabbit | Abcam | ab75351 | 1/25 |
| Cilp | Rabbit | biorbyt | orb182643 | 1/100 |

### 1.8 Ultrasound Analysis of Cardiac Function

The mice are anaesthetized by inhalation of isoflurane and placed on a heating platform (VisualSonics). Temperature and heart rate are continuously monitored. The image is taken by a Vevo 770 high-frequency echocardiograph (VisualSonics) with 707B probe. Ultrasound measurements in 2D mode and M mode (motion) are taken along the large and small parasternal axis at the widest level of the left ventricle. Quantitative and qualitative measurements are performed using the Vevo 770 software. The mass of the left ventricle is estimated using the following formula:

Mass of the left ventricle (g)=0.85(1.04(((diameter of the left ventricle at the end of diastole+thickness of the intraventricular septum at the end of diastole+thickness of the posterior wall at the end of diastole)³−diameter of the ventricle at the end of diastole3)))+0.6.

For each ultrasound of a mouse heart, about 5 measurement points are taken. The measuring point corresponding to the maximum size of the left ventricle in diastole is then used, as it represents the maximum dilatation that the mouse heart can reach.

### 1.9. Construction of AAV Transfer Plasmids

AAV plasmid vectors comprising an expression cassette for expressing whole candidate human proteins (CILP, DKK3, SFRP2 and CCN5/WISP2) flanked by two AAV2 ITRs were ordered from Genewiz. The expression cassette comprises the coding sequence preceded by a chimeric intron under control of human cardiac troponin T promoter (hTNNT2) and SV40 polyadenylation signal. Coding sequence for hCILP is the nucleotide sequence GenBank/NCBI accession number NM_003613.4 as accessed on 25 Apr. 2020 or SEQ ID NO: 1 coding for hCILP protein of SEQ ID NO: 2). Coding sequence for hDKK3 is the nucleotide sequence GenBank/NCBI accession number NM_015881.5 as accessed on 27 Apr. 2020 or SEQ ID NO: 3 coding for hDKK3 protein of SEQ ID NO: 4. Coding sequence for hSFRP2 is the nucleotide sequence GenBank/NCBI accession number NM_003013.3 as accessed on 31 May 2020 or SEQ ID NO: 5 coding for hSFRP2 protein of SEQ ID NO: 6. Coding sequence for CCN5/WISP2 is the nucleotide sequence GenBank/NCBI accession number NM_003881.3 as accessed on 3 May 2020 or SEQ ID NO: 7 coding for hCCN5WISP2 protein of SEQ ID NO: 8. AAV transfer vectors were constructed by inserting the different expression cassettes between two AAV2 ITRs.

The shRNA plasmid construct for LTBP2 gene inhibition has been ordered from Vigene Bioscience. The construct comprises 4 individual shRNA sequences targeting different sequences of human LTBP2 transcript expressed from two sets of H1 and hU6 promoters in opposite orientation and the GFP reporter gene under the control of the CMV promoter. The shRNA plasmid construct is flanked by 2 AAV2 ITRs. The sequences selected for LTBP2 gene are described in Table 5.

TABLE 5
shRNA sequences

| shRNA sequences | SEQ ID NO |
|---|---|
| GGAAGTCTAGTGACCAGAATA | 11 |
| GCTGGTGAAGGTGCAAATTCA | 12 |
| GCTTCTATGTGGCGCCAAATG | 13 |
| GCACCAACCACTGTATCAAAC | 14 |

### 1.10. Production of Plasmids

Plasmids are produced by transforming 45 μL of DH10B bacteria with 2 μL of plasmid. Thermal shock is achieved by alternating 5 minutes in ice, 30 seconds at 42° C. and cooling on ice. Then, 250 μL of SOC (super optimal broth) medium is added before incubation at 37° C. for 1 h under agitation. The bacteria thus transformed are isolated by a 50 μL culture over night at 37° C. on a box of LB (lysogeny broth) containing ampicillin in order to select the bacteria having integrated the plasmid. A clone is transplanted the next day for a pre-culture of a few hours at 37° C. in 3 mL of LB medium containing antibiotic. Samples are kept for freezing in 50% glycerol. An overnight culture is then performed in 2 L Erlenmeyer containing 500 mL of antibiotic-containing medium and 1 mL of the preculture at 37° C. A NucleoBond PC 2000 EF (Macherey Nagel) kit is then used according to the supplier's instructions to purify the plasmids which are then sterilized by filtration at 0.22 μm and assayed with Nanodrop.

An enzymatic digestion is performed to check the plasmid with the restriction enzymes SMA1 and NHE1. A mixture containing 1 μg of DNA, 2 μL of buffer fast digest green 10×, 1 μl of each enzyme in sterile water for a total amount of 20 μl is stirred for 20 min at 37° C. A 1% agarose gel in TAE (Tris, Acetate, EDTA) containing SYBR™ Safe DNA Gel Stain (Invitrogen) is poured before depositing the digest products and the size marker O'GeneRuler™ DNA Ladder mix.

### 1.11 Viral Vector Production

The tri-transfection method is used to prepare recombinant viruses. HEK293 cells are used as packaging cells to produce the virus particles. Three plasmids are required: the vector plasmid, which provides the gene of interest, the helper plasmid pAAV2-9_Genethon_Kana (Rep2Cap9), which provides the Rep and Cap viral genes, and plasmid pXX6, which contains adenoviral genes and replaces the co-infection by an adenovirus, necessary for AAV replication. The cells are then lysed and the viral particles are purified. Vectors are produced in suspension.

Cell inoculation (day 1): Use of HEK293T clone 17 cells at confluence, inoculated in 1 L agitation flasks: 2E5 cells/mL in 400 mL of F17 medium (Thermo Fisher scientific). Incubation under agitation (100 rpm) at 37° C.-5% CO₂-humid atmosphere.

Cell Transfection (Day 3): Cells are counted and cell viability is measured on Vi-CELL after 72 h of culture. The transfection mix is prepared in Hepes buffer at 10 mg/mL for each plasmid according to its concentration, size and the amount of cells in the flask, the ratio of each plasmid is 1. Incubation 30 minutes at RT after the addition of transfection agent and homogenization of the solution. The transfection mixture and 3979 μL of culture medium (F17 GNT Modified) are transferred to shaker flasks containing 400 mL of culture which are incubated under agitation (130 rpm) at 37° C.-5% CO2-wet atmosphere. After 48 h, treatment of the cells with benzonase: dilution of Benzonase (25 U/mL final) and MgCl2 (2 mM final) in F17 medium, addition of 4 mL per flask.

Viral vector harvest (day 6): Cells are counted and cell viability is measured on Vi-CELL, then 2 mL of triton X-100 (Sigma, 1/200th dilution) are added before incubating 2.5 hours at 37° C. with agitation. The erlenmeyers are transferred to Corning 500 mL and centrifuged at 2000 g for 15 minutes at 4° C. Supernatants are transferred to new Corning 500 mL before adding 100 mL of PEG 40%+NaCl and incubating 4 h at 4° C. The suspension is centrifuged at 3500 g for 30 minutes at 4° C. The pellets are resuspended in 20 mL TMS at pH 8 (Tris HCl at 50 mM, NaCl at 150 mM and MgCl2 at 2 mM, diluted in water) and transferred to Eppendorf 50 mL before the addition of 8 μL benzonase. After 30 min incubation at 37° C., the tubes are centrifuged at 10,000 g for 15 min at 4° C.

Cesium Chloride Gradient Purification: To achieve the gradient, 10 mL of cesium chloride at a density of 1.3 grams/mL is deposited in ultracentrifuge tubes. A volume of 5 mL of cesium chloride at a density of 1.5 grams/mL is then placed underneath. The supernatant is gently deposited on top of the cesium chloride and the tubes are ultracentrifuged at 28,000 RPM for 24 hours at 20° C. Two bands are observed: the upper band contains the empty capsids and the lower band corresponds to the full capsids. Both strips are collected avoiding the removal of impurities. The sample is mixed with cesium chloride at a density of 1.379 g/mL in a new ultracentrifuge tube and then ultracentrifuged at 38,000 RPM for 72 hours at 20° C. The solid capsid strip is removed.

Concentration and filtration: The removal of cesium chloride from the viral preparation and the concentration are carried out on Amicon® (Merck) filters. On Amicon® (Merck) filters, the vectors are concentrated by ultrafiltration with a cut-off of 100 kDa. Amicon membranes are first hydrated with 14 mL 20% ethanol, centrifuged 2 min at 3000 g, then equilibrated with 14 mL PBS, centrifuged 2 min at 3000 g, and then with 14 mL 1,379 ClCs. The collected solid capsid strip is placed on the filters and centrifuged 4 min at 3000 g. 15 mL PBS 1×+F68 formulation buffer is added, before further filtration 2 min at 1500 g. The three previous steps are repeated 6 more times before recovering the last concentrate. The samples are then filtered at 0.22 μm.

Titration: The vector is then assayed by quantitative PCR.

### 1.12 Mice Treatment

1-month-old mice (DeltaMex5) and DBA/2J-mdx (DBA2mdx) strains, and their respective controls, strains C57BL/6 and DBA/2) were injected intravenously at a dose of ^{2e11} vg/mouse (equivalent to a dose of 1e13 vg/kg for a mouse of approximately 20 g) of AAV vector or by PBS. After 3 months of vector expression, the hearts of the mice were ultrasonographed prior to collection. The overall, histological and functional consequences on the heart were then studied. The mice used in this study were male titin^{Mex5-/Mex5-}

### 1.13 Statistics

In all statistical analyses, the differences are considered significant at P<0.05 (*), moderately significant at P<0.01 (**) and highly significant at P<0.001 (***), with P=probability. Bar graphs are shown as means+SEM standard deviations. The graphs are made using the GraphPad software.

Analysis of the distribution of fibrosis over the whole heart: In order to ensure that the fibrosis is homogeneous in the heart (H0 hypothesis), we randomly drew 20 values from the 483 fibrosis ratio values. These values were compared 10 to 10 with a Wilcoxon test (Software R) to obtain a p-value. This operation was repeated 1000 times, resulting in 1000 p-values. Among these values some are below 0.05 showing that in some cases our hypothesis of fibrosis invariance is not valid. Out of the 1000 statistical tests, we counted how many gave a value below 0.05. We repeated the entire process 100 times to obtain an average of the percentage for which our H0 hypothesis is false. This average is 4%. This means that our hypothesis is valid 96% of the time, and therefore corresponds to an overall p-value of 0.04, which is statistically acceptable.

Ultrasound analysis: In order to determine the relationships between the parameters and which parameters are of interest for the study, the statistical software R is used. The scatterplotMatrix function was used to visualize the correlations between the measurements at different ages and to select the parameters to be studied. The statistical analyses are performed with Rcmdr and the graphs with the GraphPad software.

### 2. Results

The inventors wanted to determine whether there were common gene expression modifications between two cardiomyopathy models: the DeltaMex5 model and the DBA/2-mdx model as well as the age at which these deregulations are established and their specificity. To do so, the inventors conducted a comparative study of transcriptome at different ages.

### 2.1 RNAseq Analysis of the Two Models of Cardiomyopathy

Total RNAseq (RNAseq) sequencing analysis was performed on heart samples from DeltaMex5 and DBA/2-mdx mice and their controls at early and late age of cardiac involvement. For DeltaMex5 mice, ages of 1 and 4 months were chosen, and for DBA/2-mdx mice, ages of 1 and 6 months. The main aim here was to identify genes present when the pathology is established that would be common to both cardiomyopathy models.

The sequencing was done according to the Illumina protocol. The differential expression of genes for each sample is calculated in relation to its control from their read number (>10). The expression difference values (or fold change) are expressed in binary logarithm (log 2.FC) and are associated with their adjusted Pvalue padj. Genes expressed significantly differentially between different conditions are determined by a log 2.FC>10.51 and a padj<0.05.

The volcano plot of the RNAseq data allows visualization for each condition of the distribution of genes and the extent of gene deregulation in the heart, as well as the extent of gene expression. The list of the 30 most deregulated (overexpressed) genes at 4 months in the heart of DeltaMex5 model is presented in the Table 6.

TABLE 6
Top 30 most deregulated (overexpressed) genes in
the heart of the DeltaMex5 model at 4 months.

| | | | Average | Average |
|---|---|---|---|---|
| Gene | log2FC | padj | DeltaMex5 | C57BL/6 |
| Spp1 | 6.60 | 5.28E−128 | 2162.74 | 6.65 |
| Gm42793 | 4.82 | 3.66E−46 | 212.55 | 0.00 |
| Cilp | 4.77 | 4.70E−278 | 3357.31 | 109.77 |
| Ltbp2 | 4.74 | 2.97E−174 | 2206.18 | 68.03 |
| Gpnmb | 4.68 | 1.57E−97 | 764.57 | 19.98 |
| Sprr1a | 4.33 | 1.41E−36 | 222.75 | 1.39 |
| Tnc | 4.28 | 2.99E−33 | 4363.96 | 38.66 |
| Gm6166 | 4.24 | 5.99E−39 | 171.45 | 2.01 |
| 8030451A03Rik | 4.22 | 4.64E−35 | 206.04 | 1.72 |
| D030025P21Rik | 4.02 | 5.44E−36 | 153.86 | 2.87 |
| Timp1 | 3.98 | 5.70E−52 | 625.54 | 24.25 |
| Col12a1 | 3.82 | 2.14E−60 | 989.43 | 50.08 |
| Col8a2 | 3.74 | 3.28E−40 | 247.08 | 10.59 |
| Sfrp2 | 3.62 | 2.08E−55 | 501.17 | 30.20 |
| Thbs4 | 3.61 | 3.79E−121 | 919.77 | 66.85 |
| Ptn | 3.40 | 1.25E−35 | 290.91 | 17.60 |
| Postn | 3.35 | 1.65E−21 | 12040.11 | 414.51 |
| Mfap4 | 3.31 | 3.99E−57 | 428.05 | 35.05 |
| Piezo2 | 3.27 | 2.87E−31 | 209.01 | 13.45 |
| Gm26771 | 3.26 | 9.27E−27 | 132.29 | 7.36 |
| Col3a1 | 3.22 | 1.72E−61 | 40685.43 | 3682.59 |
| Col14a1 | 3.20 | 9.47E−116 | 2081.22 | 207.54 |
| Ctss | 3.19 | 3.04E−59 | 1773.00 | 162.81 |
| Trem2 | 3.16 | 2.89E−28 | 259.21 | 17.93 |
| Atp6v0d2 | 3.15 | 2.67E−17 | 58.07 | 0.67 |
| Apol7d | 3.15 | 5.17E−32 | 541.35 | 41.67 |
| AC125167.1 | 3.12 | 3.11E−46 | 1533.67 | 141.84 |
| Lgals3 | 3.10 | 6.97E−19 | 747.38 | 35.14 |
| Mpeg1 | 3.09 | 8.80E−23 | 2205.83 | 143.04 |
| Dkk3 | 3.01 | 2.78E−32 | 299.63 | 27.12 |

Underlined = model specific

For the DBA/2-mdx model, the list of the 30 most deregulated (overexpressed) genes in the heart at 6 months is presented in the Table 7.

TABLE 7
Top 30 most deregulated (overexpressed) genes in the
heart of the DBA/2-mdx model at 6 months.

| | | | Average | Average |
|---|---|---|---|---|
| Gene | log2FC | padj | DBA/2-mdx | DBA/2 |
| Ighg2c | 3.99 | 2.34E−40 | 127.71 | 0.00 |
| Tnc | 3.76 | 1.89E−111 | 1637.90 | 96.86 |
| Cilp | 3.27 | 4.59E−70 | 760.53 | 62.18 |
| Sprr1a | 3.02 | 7.35E−22 | 75.33 | 1.37 |
| Mt2 | 2.98 | 3.69E−44 | 425.98 | 39.53 |
| Timp1 | 2.83 | 1.53E−19 | 735.88 | 34.09 |
| 8030451A03Rik | 2.65 | 5.15E−18 | 77.45 | 4.44 |
| Serpina3n | 2.60 | 9.78E−18 | 2728.99 | 200.98 |
| Chile1 | 2.54 | 1.01E−18 | 173.35 | 15.73 |
| Hamp2 | −2.51 | 2.58E−38 | 61.43 | 427.96 |
| Lrp8 | 2.47 | 1.81E−16 | 132.79 | 11.37 |
| Saa3 | 2.41 | 6.08E−13 | 46.74 | 0.65 |
| Fam46b | 2.36 | 5.00E−34 | 511.91 | 83.18 |
| Per2 | 2.35 | 8.14E−32 | 292.28 | 47.22 |
| Fgl2 | 2.34 | 4.46E−65 | 3216.00 | 585.39 |
| Lox | 2.32 | 7.38E−52 | 919.81 | 166.22 |
| Crlf1 | 2.30 | 1.98E−19 | 172.27 | 24.17 |
| Postn | 2.28 | 6.18E−21 | 9086.68 | 1378.81 |
| Ereg | 2.27 | 3.18E−12 | 58.87 | 3.76 |
| Cfb | 2.27 | 4.86E−41 | 632.26 | 115.30 |
| Nxpe5 | 2.27 | 4.06E−28 | 215.52 | 36.33 |
| Gm20547 | 2.27 | 6.53E−48 | 712.93 | 133.02 |
| Ccl6 | 2.26 | 1.05E−64 | 1020.70 | 197.87 |
| Ccl9 | 2.24 | 4.79E−43 | 492.52 | 93.30 |
| Pak3 | 2.20 | 3.81E−15 | 117.72 | 15.86 |
| Mmp3 | 2.17 | 7.28E−35 | 967.23 | 187.88 |
| Srpx | 2.17 | 9.38E−31 | 366.18 | 70.08 |
| Clec4d | 2.16 | 1.12E−12 | 66.61 | 7.53 |
| Ccl7 | 2.16 | 2.68E−18 | 140.95 | 22.98 |
| He33 | 2.15 | 2.60E−32 | 353.56 | 69.27 |

Underlined = model specific

The Top 30 most increased genes in the heart of the DeltaMex5 model at 4 months include genes of the WNT and TGF-β signaling pathways. In particular, two genes that belong directly to the WNT signaling pathway are overexpressed: SFRP2, coding for Secreted Frizzled-Related Protein-2 (log 2FC=3.62, P=2.08E-55) and DKK3 coding for Dickkopf related protein-3 (log 2FC=3.01, P=2.78E-32). Two genes that belong directly to the TGF-β WNT signaling pathway are also overexpressed: the CILP gene, coding for Cartilage Intermediate Layer Protein (log 2FC=4.77, P=4.70E-278), a negative regulator of the TGF-β pathway (Shindo et al. Int. Journal of Gerontology, 2017, 11, 67-74) and LTBP2 (log 2FC=4.74, P=2.97E-174) coding for the Latent-Transforming growth factor Beta-binding Protein 2, a modulator of the TGF-β pathway (Sinha et al., Cardiovascular Research, 2002, 53, 971-983). In the DBA/2-mdx model at 6 months, the inventors find in the first 5 positions CILP gene as one of the most deregulated gene. At 1 month, the number of deregulated genes is much smaller and the deregulated genes are deregulated to a lesser extent with a maximum log 2FC of 1.

The Venn diagram representation of RNAseq results allows the visualization of the numbers of common or specific deregulated genes in a model or a stage of disease progression. Of the 46,717 genes included in the RNAseq analysis, 4,850 genes were found to be significantly deregulated (Ilog2FCI>0.5 and pvalue<0.05) in either model at early or late age of cardiac involvement compared to control. At an early age, the heart of DeltaMex5 mice has only 44 deregulated genes, whereas the heart of DBA/2-mdx mice already has 2,186, with only 4 genes in common in both models. At a later age, the DeltaMex5 heart has 2,621 deregulated genes and the DBA/2-mdx heart has 2,202, of which 1,175 are common to both models, of which 708 genes are specific for the advanced age of cardiomyopathy. Only 9 genes are specific for the DeltaMex5 model, while 232 are specific for the DBA/2-mdx model. Of all the deregulated genes, a greater proportion of the genes are over-expressed rather than under-expressed. The majority of the most over-expressed genes are common between the two models. However, genes deregulated in the hearts of DeltaMex5 mice at 4 months are more strongly deregulated than genes deregulated in the hearts of DBA/2-mdx mice at 6 months (log 2FC maximum of 4 versus 6.6). It was also observed that, although the cardiac involvement between the two models was different, the transcriptional deregulations associated with them mostly involved the same genes and signaling pathways at a late stage.

To complete this analysis, the Ingenuity Pathway Analysis (IPA, Qiagen) software, which uses a repository of biological interactions and functional annotations to help interpret the data into biological mechanisms was used. At one month of age, no increase in signaling pathways was identified in the hearts of DeltaMex5 and DBA/2-mdx mice. Analysis by IPA allowed to highlight the biological functions whose genes are most represented in the deregulated genes in an advanced phase. In first position in both models, more than 150 genes involved in cardiovascular disease were found in the RNAseq analysis. In second position, more than 150 deregulated genes are categorized in the family of lesions and abnormalities on an organ. Finally, in third position, nearly 200 genes related to the function and development of the cardiovascular system were found.

The inventors also used another function of the IPA software to determine the toxicity associated with the observed changes in gene expression, and this only in the advanced phases. Many deregulated genes were identified: 86 genes associated with cardiac enlargement in the DeltaMex5 model and 85 in the DBA/2-mdx model, 45/48 genes that could lead to cardiac dysfunction, 38/36 genes in cardiac dilatation, 27/28 genes in cardiac fibrosis and 35/37 in cardiac necrosis.

The PANTHER gene ontology classification system was also used to determine the most deregulated signalling pathways in the late-stage models. In both models, the perturbations appear to be very similar as seen in the analysis of the Venn Diagrams. In the late-stage models, the WNT signaling pathway is found in 4^{th} position of the most deregulated signaling pathways in the heart of DeltaMex5 mice and in 3^{rd} position in the heart of DBA/2-mdx mice. A total of 40 genes belonging to this pathway are deregulated (overexpressed), including SFRP2 and DKK3. The TGF-β pathway, is found in 22^{nd} and 19′ position in the heart of DeltaMex5 and DBA/2-mdx, with more than 15 deregulated genes, to which CILP and LTBP2, two of the most over-expressed genes, belong.

### 2.2 Validation of Deregulated Genes

The deregulation of CILP-1, DKK3, SFRP2, LTBP2 some of the most deregulated genes was evaluated under different conditions. WISP2 from the Wnt pathway was also selected.

None of these genes are overexpressed in the DeltaMex5 model at 1 month, while DKK33 is already overexpressed in the DBA2-mdx model. All genes are over-expressed in the later age of the disease (Table 8).

TABLE 8
Deregulation of the genes of interest in the models

| | | C2orf40 | CILP | COMP | DIO2 | DKK3 | LTBP2 |
|---|---|---|---|---|---|---|---|
| DeltaMex5 | log2FC | 0.12 | 0.16 | 0.09 | 0.22 | 0.11 | 0.11 |
| 1 month | padj | 0.00E+00 | 0.00E+00 | 0.00E+00 | 8.97E−01 | 0.00E+00 | 0.00E+00 |
| | Average | 18 | 560 | 106 | 60 | 180 | 388 |
| | DeltaMex5 | | | | | | |
| | Average | 7 | 136 | 41 | 33 | 49 | 91 |
| | C57BL/6 | | | | | | |
| DeltaMex5 | log2FC | 1.60 | 4.77 | 2.94 | 2.03 | 3.01 | 4.74 |
| 4 months | Padj | 4.08E−05 | 4.70E−278 | 4.48E−15 | 1.08E−13 | 2.80E−32 | 3.00E−174 |
| | Average | 32 | 3357 | 990 | 130 | 300 | 2206 |
| | DeltaMex5 | | | | | | |
| | Average | 6 | 110 | 37 | 25 | 27 | 68 |
| | C57BL/6 | | | | | | |
| DBA/2-mdx | log2FC | 1.28 | 0.63 | 0.73 | 0.58 | 1.06 | 0.79 |
| 1 month | padj | 0.01 | 2.30E−01 | 1.37E−01 | 2.64E−01 | 2.10E−02 | 9.00E−02 |
| | Average | 80 | 417 | 211 | 68 | 936 | 1559 |
| | DBA/2-mdx | | | | | | |
| | Average DBA/2 | 10 | 111 | 35 | 38 | 53 | 160 |
| DBA/2-mdx | log2FC | 1.19 | 3.27 | 1.59 | 1.49 | 1.37 | 1.94 |
| 6 months | padj | 1.14E−03 | 4.60E−70 | 7.35E−06 | 9.85E−08 | 1.60E−04 | 1.00E−10 |
| | Average | 47 | 761 | 558 | 123 | 542 | 1688 |
| | DBA/2-mdx | | | | | | |
| | Average DBA/2 | 12 | 62 | 49 | 33 | 75 | 266 |

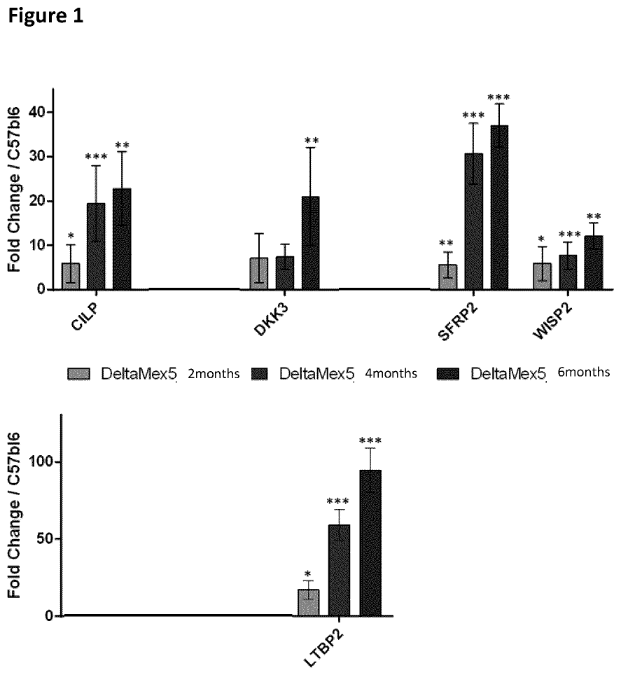

Validation of RNAseq data was then performed on hearts of the DeltaMex5 model at different ages (2, 4 and 6 months) by an individual qPCR to confirm their overexpression and assess their modification over time. All genes are significantly overexpressed from 2 months in the model, except DKK3 and gene overexpression increases progressively with age (FIG. 1).

The RNAseq analysis shows that Wnt and TGF-β pathways are both impaired and their genes, in particular CILP-1, DKK3, SFRP2 and LTBP2 are overexpressed in two models of genetically-induced dilated cardiomyopathies, Duchenne muscular dystrophies (DBA2mdx mice) and titinopathies (DeltaMex5 mice). Overexpression of the selected genes (CILP-1, DKK3, SFRP2, LTBP2 and WISP2) was validated by qPCR analysis. These results prompted the inventors to assess the effect of the modulation of the Wnt and TGF-β pathways, on the cardiac phenotype of the model, in particular by modulating the expression of the CILP-1, DKK3, SFRP2 and LTBP2 genes using a gene transfer approach.

### 2.3 Modulation of WNT and TGF-β Pathways by Gene Transfer Approach

The inventors, then wanted to assess the impact of modulation of the WNT and TGF-β pathways on the cardiac phenotype of the model. They chose to study the modulation of several genes belonging to the WNT and TGF-β pathways, either by overexpression or by inhibition using gene transfer strategies.

For the gene transfer approaches, several candidates were selected from the most deregulated genes: CILP and LTBP2 which belong to the TGF-β pathway, and WISP2/CCN5, DKK3 and SFRP2 which belong to the WNT pathway. AAV serotype 9 was chosen since it is described as having a significant cardiac tropism (Zincarelli et al., Molecular Therapy, 2008, 16, 1073-1080). For transgene expression, the chosen promoter is the human cardiac troponin Tnnt2 (cTnT) promoter, a cardiomyocyte-specific promoter (Wei et al., Gene, 2016, 582, 1-13). The AAV9 vector construct was validated using GFP-luciferase reporter.

Based on this GFP-luciferase construction, the inventors replaced the region coding for GFP and luciferase by the transgene of their choice preceded by a chimeric intron. The coding sequences chosen are the following human sequences: CILP (NCBI/GenBank accession number NM_003613.4) for the TGF-β pathway and DKK3 (NCBI/GenBank accession number NM_015881.5), SFRP2 (NCBI/GenBank accession number NM_003013.3) and WISP2 (NCBI/GenBank accession number NM_003881.3) for the WNT pathway.

The strategy chosen for inhibiting LTBP2 gene expression is that using shRNAs. These are small RNAs with a hairpin structure, their action is based on the principle of interfering RNA, neutralizing the messenger RNA of the target. The inventors have chosen 4-in-1 shRNAs for enhanced efficiency of transgene neutralization: four individual sh sequences are grouped together in a plasmid. The shRNAs were selected using Thermofisher's RNAi Designer tool. The 4 shRNAs with the best specific recovery score for the gene of interest were selected. They were then ordered from Vigene Bioscience, under the control of H1 and U6 ubiquitous promoters.

After in vitro and in vivo validation of the vector under consideration, an evaluation of the consequences of in vivo gene transfer on fibrotic status and cardiac function was performed on the DeltaMex5 model (the more severe model of the two). The effect of vectors expressing the human CILP genes for the TGF-β pathway and WISP2, SFRP2 and DKK3, for the WNT pathway was tested as well as shRNA inhibition vector for LTBP2. The approaches that have shown an interest in the DeltaMex5 model are currently being applied to the DBA/2-mdx model.

### 2.3.1 Modulation of the TGF-β Pathway Genes (CILP and LTBP2)

The modulation of the TGF-β pathway was tested by overexpression of CILP (vector AAV9-hTnnt2-hCILP) and inhibition of LTBP2 (AAV9-4inlshRNA-mLTBP2-GFP). 1-month-old mice were injected intravenously at a dose of ^{2e11} vg/mouse (equivalent to a dose of 1e13 vg/kg for a mouse of approximately 20 g) of AAV vector or by PBS. After 3 months of vector expression, the hearts of the mice were ultrasonographed prior to collection. The overall, histological and functional consequences on the heart were then studied.

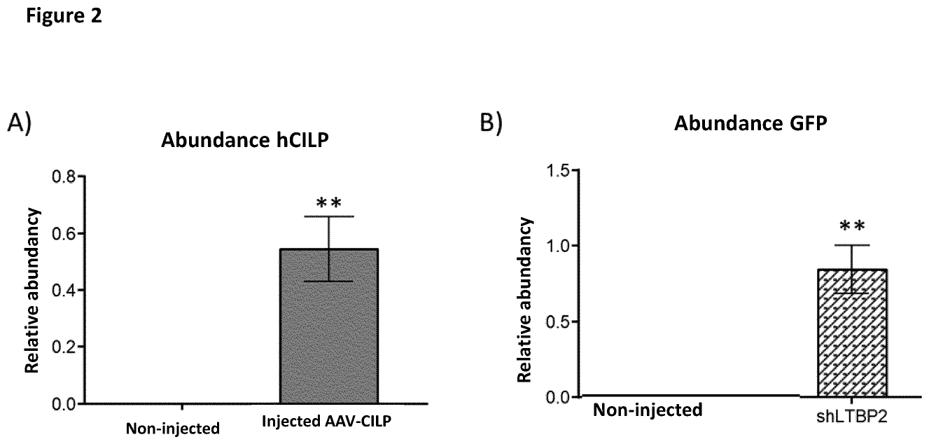

Vector expression in the heart was verified in mice injected with AAV9-hTnnt2-hCILP by assaying the relative abundance of hCILP in RT-qPCR. Since the transgene is a human transgene, it was detected only in mice injected with the vector but not in mice injected with PBS (FIG. 2A). Expression of the vector AAV9-4inlshRNA-mLTBP2-GFP is detected using the GFP reporter gene which is present only in mice injected by the vector (FIG. 2B). These RT-qPCR assays confirm the presence of the transgenes 3 months after vector injection.

### 2.3.1.1 Morphological Evaluation

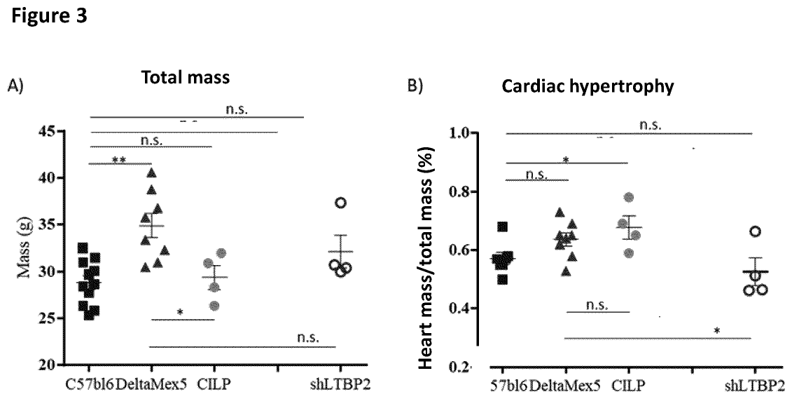

Mouse mass was significantly decreased in mice treated with the AAV9-hTnnt2-hCILP vector after 3 months (29.38±1.29 g, n=4, versus 34.9±1.3 g, n=8, P=0.024). The mass of mice treated with the AAV9-4inlshRNA-mLTBP2-GFP vector remains similar (32.13±1.76, P>0.05). The values are no longer significantly different from the mean mass of C57BL/6 mice (28.81±0.72, n=11) (FIG. 3A). Heart hypertrophy, as measured by the ratio of heart mass to total mouse mass, in mice treated with the AAV9-hTnnt2-hCILP vector remains close to DeltaMex5 mice (0.68±0.04, n=4 versus 0.63±0.02%, n=8, P>0.05), but becomes significantly increased compared to C57BL/6 mice (0.57±0.02%, n=7, P=0.027). In contrast, it was significantly decreased in mice treated with the AAV9-4inlshRNA-mLTBP2-GFP vector (0.53±0.048, P=0.03) compared to untreated DeltaMex5 mice and became comparable to C57BL/6 mice (FIG. 3B).

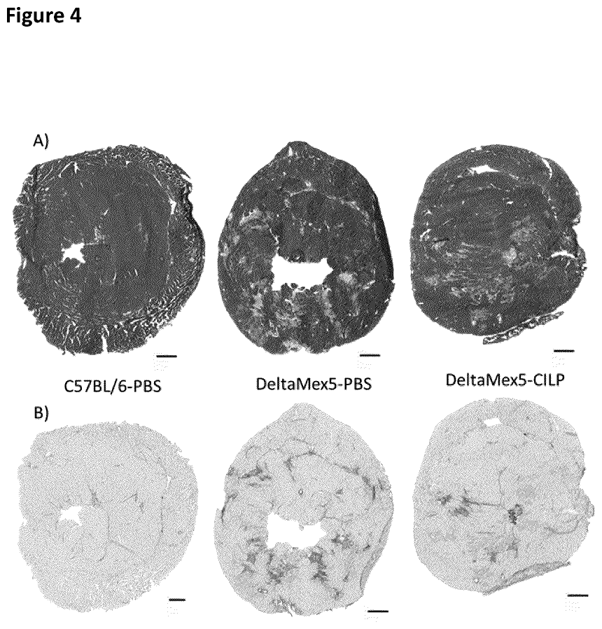

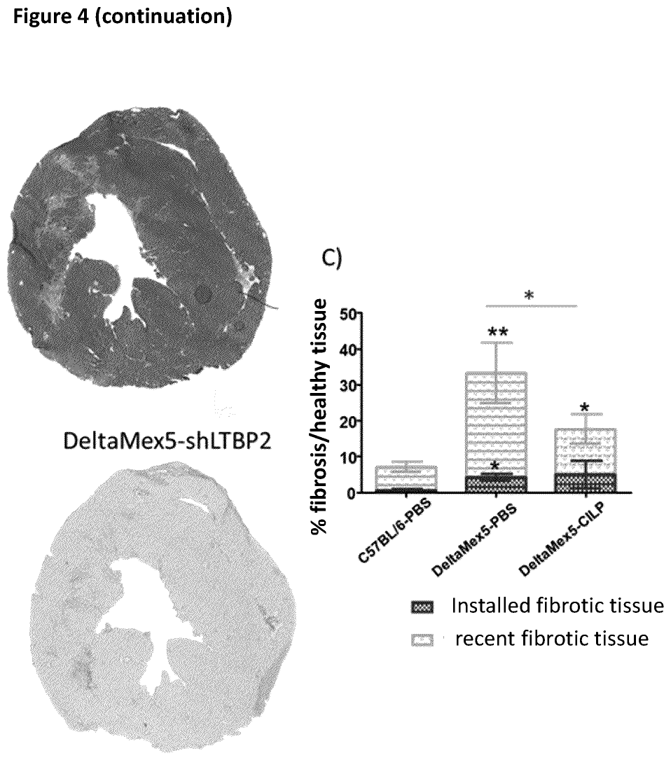

Histological analyses were then performed on the hearts of the mice. HPS staining revealed persistence of the damaged tissue in mice treated with the different vectors (FIG. 4A). Sirius Red staining still showed the presence of fibrotic tissue in the hearts of mice treated with the two vectors, but in smaller quantities compared to DeltaMex5 control mice (FIG. 4B). The quantification of fibrosis by WEKA in tissues by collagen staining with Sirius Red confirms that the overall fibrosis rate is decreased in the hearts of DeltaMex5 mice treated with AAV9-hTnnt2-hCILP compared to the hearts of DeltaMex5-PBS mice (17.59±1.27% versus 33.31±4.65%, P=0.017, n=4), although it is still higher than the fibrosis heart rate in C57BL/6 mice (7.11±0.77%, P=0.0004, n=4). Looking at the different types of fibrosis, it is observed that installed fibrosis varies little (4.14±0.45% versus 4.95±1.95%, P>0.05), but recent fibrosis is significantly less present in DeltaMex5 mice given AAV9-hTnnt2-hCILP (12.64±2.04% versus 29.17±4.24, P=0.0126) (FIG. 4C). Quantification on AAV-treated samples with shRNA has not yet been performed but observation of the slices indicates that it is decreased.

### 2.3.1.2 Functional Evaluation

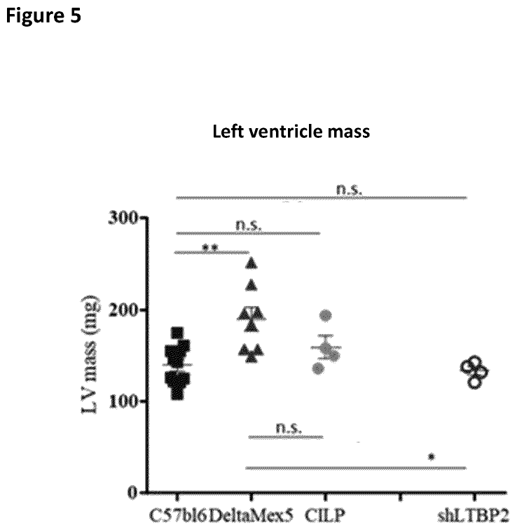

Ultrasound analyses of cardiac function were performed at 4 months, after 3 months of vector expression (FIG. 5). Among the parameters evaluated (estimated left ventricle mass and volume, ejection fraction), there was a significant variation in the estimated left ventricular mass in mice injected with AAV9-4inlshRNA-mLTBP2-GFP with a decrease of almost 40% compared to DeltaMex5 controls (133±4.74 mg, n=4, P=0.01). (127±11.05 mg,

### 2.3.1.3 Molecular Evaluation

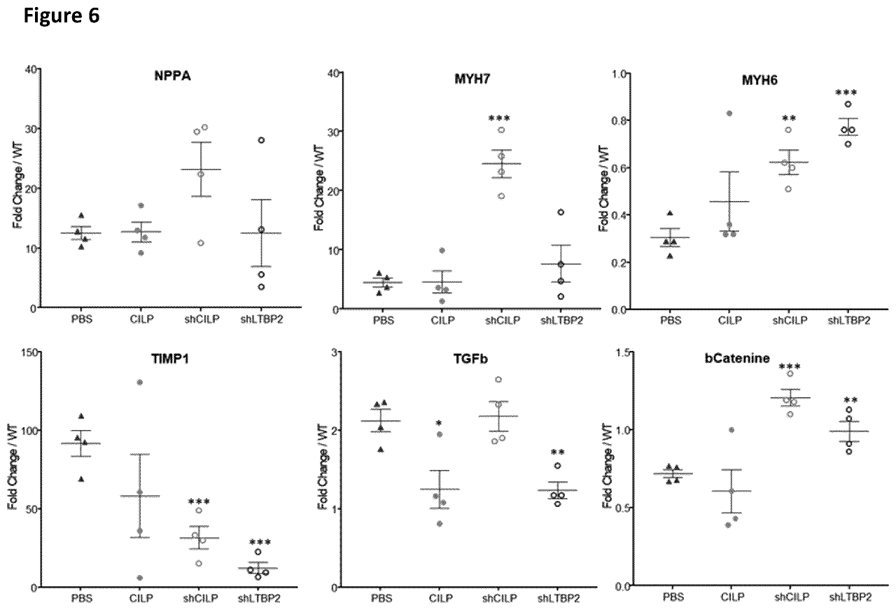

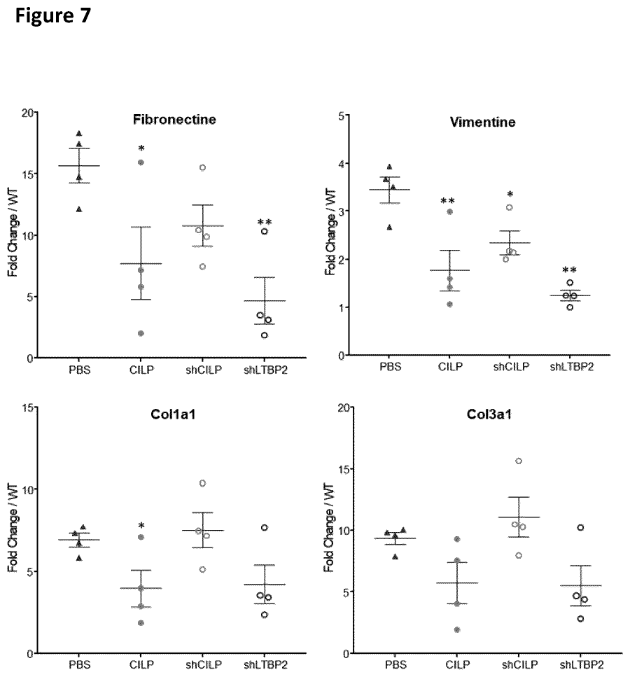

Tissue RNA markers of cardiac involvement (Nppa, Myh7, Myh6, Timp1, Tgf-β1) were measured by RT-qPCR (FIG. 6). Only Tgf-β1 was significantly decreased in mice injected with the AAV9-hTnnt2-hCILP vector compared to DeltaMex5-PBS mice with a ratio close to 1 (1.25±0.25 versus 2.13±0.14, P=0.02). Only Timp1 was significantly decreased in injected mice compared to DeltaMex5-PBS mice (31.67±6.98, P=0.001). In mice injected with the AAV9-4inlshRNA-mLTBP2-GFP vector, Myh6 and P-catenin are increased relative to uninjected mice (0.77±0.035, P=0.0001 and 0.99±0.008, P=0.008) and are normalized to C57BL/6 mice with a ratio close to 1. Conversely, TIMP1 and TGF-β3 are decreased (12.31±3.49, P=0.0001 and 1.24±0.11, P=0.002), and TGF-β is normalized with respect to C57BL/6 mice.

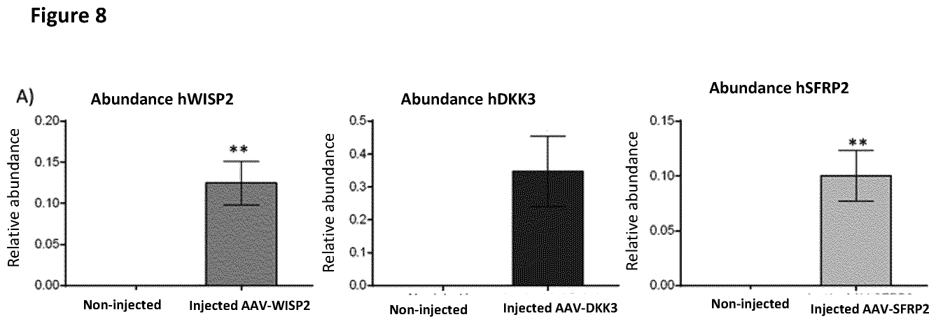

Fibrosis RNA tissue markers (Fibronectin, Vimentin, Collagen 1a1 and Collagen 3al) were also measured by RT-qPCR (FIG. 7). In mice injected with AAV9-hTnnt2-hCILP, the 3 main markers of fibrosis were significantly reduced compared to mice injected with PBS:fibronectin (7.72±2.95 versus 15.67±1.4, P=0.05), vimentin (1.77±0.43 versus 3.45±0.27, P=0.016) and collagen 1a1 (3.95±1.13 versus 6.92±0.41, P=0.049). Collagen 3al has a non-significant decreasing trend (5.72±1.68 versus 9.35±0.49, P=0.08). These results are related to the previous results: there was no improvement in cardiac function in the treated mice, but there was an improvement in fibrosis. Interestingly, for all markers, the mouse showing the best normalization of results compared to C57BL/6 mice was the mouse with the highest hCILP level. In mice injected with AAV9-4inlshRNA-mLTBP2-GFP both fibronectin and vimentin are decreased (4.68±1.91, P=0.0035 and 1.25±0.10, P=0.0003). Vimentin was normalized to C57BL/6 mice.

### 2.3.2 Modulation of the WNT Pathway Genes (WISP2, DKK3, SFRP2)

The modulation of the WNT pathway was tested by overexpression of WISP2, DKK3 and SFRP2. The 1-month-old mice were injected intravenously at a dose of 2e11 vg/mouse or by PBS. After 3 months of vector expression, the hearts of the mice were ultrasonographed prior to sampling.

Vector expression in the heart was tested in mice injected with AAV9-hTnnt2-hWISP2, AAV9-hTnnt2-hDKK3 and AAV9-hTnnt2-hSFRP2 by RT-qPCR assay of the relative abundance of hWISP2, hDKK3 and hSFRP2 respectively (FIG. 8).

### 2.3.2.1 Morphological Evaluation

The mass of mice treated with the three vectors remains similar to that of untreated DeltaMex5 mice (WISP2: 35.7±1.106; DKK3: 32.58±1.31 g; SFRP2: 32.8±1.45 g), as well as the heart hypertrophy measured by the heart mass as a proportion of the total mass of the mouse (WISP2: 0.55±0.02%; DKK3: 0.68±0.05%; SFRP2: 0.64±0.06%).

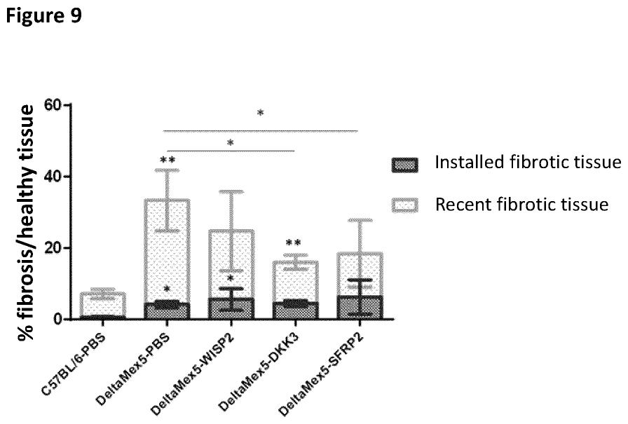

Histologically, the HPS stain reveals persistence of the damaged tissue in mice treated with the three vectors. Sirius Red staining still shows the presence of fibrotic tissue in the hearts of treated mice, but appears weaker for the AAV9-hTnnt2-DKK3 and SFRP2 vectors. The quantification of fibrosis by WEKA in tissues by Sirius Red collagen staining shows that for the AAV9-hTnnt2-hDKK3 and SFRP2 vectors, the overall fibrosis rates are decreased (DKK3: 17.31±4.79%; SFRP2: 18.38±3.69% versus 33.31±4.65%, P=0.01 and P=0.046 n=4), and that this is due to a decrease in recent fibrosis only (DKK3: 11.5±1.01%; SFRP2: 12.18±4.66% versus 29.17±4.24%, P=0.007 and P>0.035 n=4) (FIG. 9).

### 2.3.2.2 Functional Evaluation

Ultrasound analyses of cardiac function and ventricular dilatation showed no change in parameters in mice injected with AAV9-hTnnt2-hWISP2, DKK3 and SFRP2 compared to mice injected with PBS. Only the left ventricular mass was significantly decreased by 30% in mice injected with the AAV9-hTnnt2-DKK3 vector (133.34±8.44 mg, n=4, versus 190±12.77 mg, n=8, P=0.01).

### 2.3.2.3 Molecular Evaluation

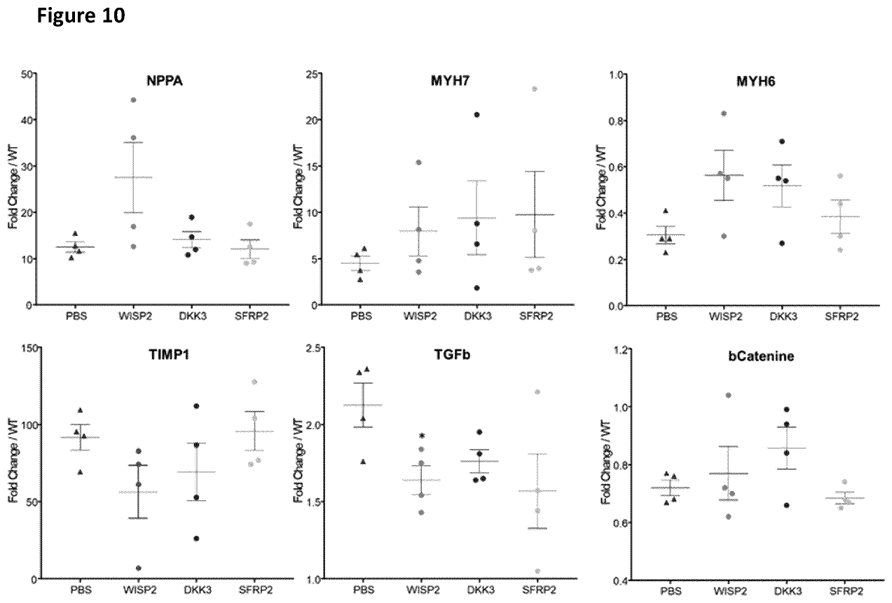

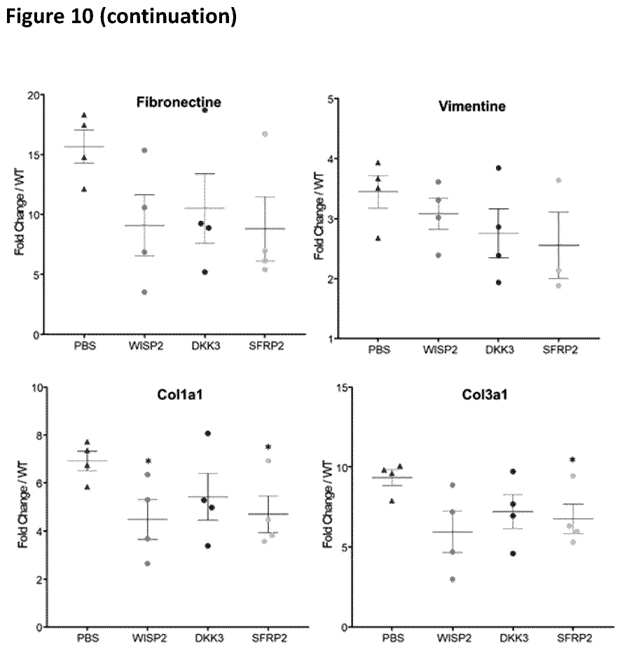

The markers of cardiac damage and fibrosis evaluated in RT-qPCR for the hearts of mice injected with AAV9-hTnnt2-hWISP2 are modified only for TGF-β and collagen 1a1 which are decreased compared to mice injected with PBS (1.64±0.94, P=0.03 and 4.49±0.83, P=0.04) (FIG. 10), the other markers are not modified (FIG. 10).

It is interesting to observe that for all markers, the mouse showing the best restoration of ratios relative to C57BL/6 mice is the mouse with the highest hWIPS2 level. No markers were significantly altered in the hearts of mice injected with AAV9-hTnnt2-DKK3. In the hearts of mice injected with AAV9-hTnnt2-hSFRP2, the only differences observed were a decrease in collagen 1a1 and 3al in the hearts of (4.70±0.77, P=0.043 and 6.76± to 0.77, P=0.048). Again it is observed that for all markers, the mouse showing the best restoration of ratios compared to C57BL/6 mice is the mouse with the highest transgene level.

### CONCLUSION

The modulation of the WNT or TGF-β pathway by surexpression of the WNT pathway genes WISP2, DKK3, SFRP2 and the TGF-β pathway gene CILP-1 or inhibition of expression of the TGF-β pathway LTBP2 gene improve tissue fibrosis after 3 months of treatment. There was no improvement in cardiac function in the treated mice, but there was an improvement in fibrosis.

1-15. (canceled)

16. A method of treating genetic dilated cardiomyopathies in a patient in need thereof, comprising administering to the patient a therapeutically effective amount of an expressible modulator of the Wnt or TGF-β pathway

17. The method according to claim 16, wherein the modulator modulates the activity of a target protein of the Wnt or TGF-β pathway and is selected from the group consisting of: aptamer, antibody, recombinant target protein, inhibitory peptide, fusion protein, decoy receptor, soluble protein and dominant negative mutant.

18. The method according to claim 16, wherein the modulator modulates the expression of a target gene of the Wnt or TGF-β pathway and is selected from the group consisting of: interfering RNA molecule, ribozyme, genome or epigenome editing enzyme complex, and target transgene.

19. The method according to claim 16, wherein the modulator is an inhibitor or activator of the Wnt pathway or an inhibitor of the TGF-β pathway.

20. The method according to claim 16, wherein the modulator is an activator of CILP-1, CCN5/WISP2, DKK3 or SFRP2, or an inhibitor of LTBP2.

21. The method according to claim 20, wherein the inhibitor of LTBP2 is an interfering RNA which specifically decreases LTBP2 expression.

22. The method according to claim 20, wherein the inhibitor of LTBP2 is a shRNA comprising at least one sequence selected from the group consisting of SEQ ID NO: 11 to 14.

23. The method according to claim 20, wherein the activator is a transgene encoding CILP-1, DKK3, SRFP2, or CCN5/WISP2 protein or a variant thereof.

24. The method according to claim 20, wherein the activator is a CILP-1, DKK3, SRFP2, CCN5/WISP2 protein or a variant thereof comprising a sequence selected from the group consisting of the sequences SEQ ID NO: 2, 4, 6 and 8 and the sequences having at least 85% identity with any one of said sequences.

25. The method according to claim 20, wherein the modulator is inserted into a nucleic acid construct comprising a cardiac promoter selected from the group consisting of: human cardiac troponin T promoter (TNNT2), alpha myosin heavy chain promoter (α-MHC), myosin light chain 2v promoter (MLC-2v), myosin light chain 2a promoter (MLC-2a), CARP gene promoter, alpha-cardiac actin promoter, alpha-tropomyosin promoter, cardiac troponin C promoter, cardiac myosin-binding protein C promoter, sarco/endoplasmic reticulum Ca²⁺ ATPase (SERCA) promoter, desmin promoter, MH promoter, CK8 promoter and MHCK7 promoter.

26. The method according to claim 20, wherein the modulator is inserted into a nucleic acid construct comprising a human cardiac troponin T promoter.

27. The method according to claim 20, wherein the modulator is inserted into a nucleic acid construct that is contained in a vector for gene therapy.

28. The method according to claim 20, wherein the modulator is inserted into a nucleic acid construct that is contained in a vector for gene therapy which comprises a viral particle.

29. The method according to claim 20, wherein the modulator is inserted into a nucleic acid construct that is contained in a vector for gene therapy which comprises an adeno-associated viral (AAV) particle.

30. The method according to claim 20, wherein the modulator is inserted into a nucleic acid construct that is contained in a vector for gene therapy, and wherein the vector comprises an adeno-associated viral (AAV) particle comprising capsid protein(s) derived from AAV serotypes selected from the group consisting of: AAV-1, AAV-6, AAV-8, AAV-9 and AAV9.rh74 serotypes.

31. The method according to claim 20, wherein the modulator is inserted into a nucleic acid construct that is contained in a vector for gene therapy, and wherein the vector comprises an adeno-associated viral (AAV) particle comprising capsid protein(s) derived from AAV9.rh74 serotype.

32. The method according to claim 16, wherein the genetic cardiomyopathy is caused by mutation in a gene selected from the group consisting of: laminin, emerin, fukutin, fukutin-related protein, desmocollin, plakoglobin, ryanodine receptor 2, sarcoplasmic reticulum Ca(2+) ATPase 2 isoform alpha, phospholamban, lamin A/C, dystrophin, telethonin, actinin, desmin, cardiac actin, sarcoglycans, titin, cardiac troponin, myosin, RNA binding motif protein 20, BCL2-associated athanogene 3, desmoplakin, tafazzin and sodium channels.

33. The method according to claim 16, wherein the genetic cardiomyopathy is caused by mutation in the dystrophin or titin gene.
